(19) United States
(12) Patent Application Publication
(10) Pub. No.: US 2016/0319292 A1
(43) Pub. Date: Nov. 3, 2016

(54) TRANSFORMED PLANT AND METHOD FOR PRODUCING EXUDATE CONTAINING SUGAR USING TRANSFORMED PLANT

(71) Applicants: Kumi TERADA, Toyota-shi (JP); Madoka YONEKURA, Nagoya-shi (JP); Satoshi KONDO, Miyoshi-shi (JP); Chikara OHTO, Toyota-shi (JP); Naohiro AOKI, Tokyo (JP); Ryu OHSUGI, Tokyo (JP); Tatsuro HIROSE, Joetsu-shi (JP)

(72) Inventors: Kumi TERADA, Toyota-shi (JP); Madoka YONEKURA, Nagoya-shi (JP); Satoshi KONDO, Miyoshi-shi (JP); Chikara OHTO, Toyota-shi (JP); Naohiro AOKI, Tokyo (JP); Ryu OHSUGI, Tokyo (JP); Tatsuro HIROSE, Joetsu-shi (JP)

(73) Assignees: TOYOTA JIDOSHA KABUSHIKI KAISHA, Toyota-shi, Aichi (JP); THE UNIVERSITY OF TOKYO, Bunkyo-ku, Tokyo (JP); National Agriculture and Food Research Organization, Ibaraki (JP)

(21) Appl. No.: 15/107,776

(22) PCT Filed: Dec. 25, 2014
(86) PCT No.: PCT/JP2014/084319

§ 371 Date: Jun. 23, 2016

(30) Foreign Application Priority Data
Dec. 27, 2013 (JP) 2013-273130

(51) Int. Cl. C12N 15/82; C07K 14/415

(52) U.S. Cl. CPC C12N 15/8245; C07K 14/415

(57) ABSTRACT

The production of exudate containing sugar from a plant at a high concentration is provided. It is provided by introducing a nucleic acid encoding an AtSWEET8 protein or a homologous nucleic acid of the nucleic acid and/or enhancing the expression of the protein encoded by the nucleic acid or the homologous nucleic acid.

### TECHNICAL FIELD

The present invention relates to a transformed plant that has gained an excellent trait by introduction of a given gene and a method for producing an exudate containing sugar using the transformed plant.

### BACKGROUND ART

For stable production of biofuel or bioplastics, low cost and stable supply of their raw material sugar is desired. The representative example of the raw material sugar is sugar accumulated in sugarcane. Extraction of sugar from sugarcane generally requires processes such as cutting down of sugarcane at a predetermined harvest time, crushing, pressing, concentration, and purification. Moreover, after harvest, the farmland requires management work such as maintenance of farm for new cultivation, planting, and spraying herbicides and insecticides. The production of the raw material sugar with plants such as sugarcane has been conventionally a process requiring a great deal of cost such as that for the production process and the cultivation, as described above.

Patent Literature 1 discloses a method for recovering a heterologous protein encoded by a heterologous gene from a plant transformed to express the heterologous gene. The method disclosed in Patent Literature 1 comprises collecting an exudate from a plant transformed to express a heterologous gene and recovering the heterologous protein from the collected exudate. Examples of the exudate in Patent Literature 1 include exudate from the rhizome and the guttation exuded from a plant as an exudate through the hydathode of the leaf.

Patent Literature 2 and Non Patent Literature 1 disclose transporter proteins involved in sugar transport in plant in Arabidopsis thaliana and rice (Oryza sativa). The transporter proteins disclosed in Patent Literature 2 and Non Patent Literature 1 are known as GLUE proteins or SWEET proteins. Introduction of a nucleic acid encoding a transporter protein disclosed in Patent Literature 2 and Non Patent Literature 1 into a plant may improve the amount of sugar transport to root.

Non Patent Literature 2 describes the confirmation of function of a cell membrane small molecule transporter by artificially localizing the cell membrane transporter on the endoplasmic reticulum (ER) and measuring the small molecule transporter activity of the ER. In particular, the glucose transporters GLUTs and SGLTs were localized on the ER and their original functions were speculated using FRET (Forster resonance energy transfer or fluorescence resonance energy transfer).

### CITATION LIST

### Patent Literature

### Patent Literature 1

JP Patent Publication (Kohyou) No. 2002-501755 A

### Patent Literature 2

JP Patent Publication (Kohyou) No. 2012-525845 A

### Non Patent Literature

### Non Patent Literature 1

Nature (2010) 468, 527-534

### Non Patent Literature 2

FASEB J. (2010) 24, 2849-2858

### SUMMARY OF INVENTION

### Technical Problem

As described in the foregoing, large cost of producing sugar using plants has been a big problem. The aforementioned problem may be however solved by including sugar at a high concentration in the exudate derived from a plant and collecting the exudate. Patent Literature 1 discloses the collection of a heterologous protein from exudate, but no technique to collect sugar from the exudate. Patent Literature 2 and Non Patent Literature 1 disclose the transporter proteins, designated as SWEETs, involved in sugar transportation and nucleic acids encoding them, but no relation between these transporter proteins or nucleic acids encoding them and the sugar content in the exudate.

Accordingly, in view of the circumstances described above, an object of the present invention is to provide a transformed plant that produces an exudate containing sugar at a high concentration and a method for producing sugar using the transformed plant.

### Solution to Problem

As a result of diligent studies to achieve the purpose described above, we have found that high sugar contents in exudate are achieved in the transformed plant in which a nucleic acid encoding a predetermined transporter protein involved in sugar transportation in plant is introduced and expression of the nucleic acid is enhanced, thereby completing the present invention.

(1) A transformed plant or a transformed plant cell in which a nucleic acid encoding an AtSWEET8 protein or a homologous nucleic acid of the nucleic acid is introduced and/or expression of a protein encoded by the nucleic acid or the homologous nucleic acid is enhanced.
(2) The transformed plant or transformed plant cell according to (1), wherein the nucleic acid encoding the AtSWEET8 protein is a nucleic acid encoding a protein of any of the following (a) to (c):
(a) a protein having the amino acid sequences of SEQ ID NO: 2;
(b) a protein having an amino acid sequence having an identity of 90% or more to the amino acid sequence set forth in SEQ ID NO: 2 and having transporter activity involved in sugar transportation;
(c) a protein having an amino acid sequence encoded by a polynucleotide hybridizable with all or a part of a polynucleotide having the nucleotide sequence set forth in SEQ ID NO: 1 under stringent conditions and having transporter activity involved in sugar transportation.
(3) The transformed plant or transformed plant cell according to (1), wherein the homologous nucleic acid is a nucleic acid encoding a protein according to any of the following (a) to (c):
(a) a protein having an amino acid sequence set forth in SEQ ID NO: 5 or 7;
(b) a protein having an amino acid sequence having an identity of 90% or more to an amino acid sequence set forth in SEQ ID NO: 5 or 7 and having transporter activity involved in sugar transportation;
(c) a protein having an amino acid sequence encoded by a polynucleotide hybridizable with all or a part of a polynucleotide having a nucleotide sequence set forth in any of SEQ ID NOs: 40 to 43 under stringent conditions and having transporter activity involved in sugar transportation.
(4) The transformed plant or transformed plant cell according to (1), wherein the homologous nucleic acid is a nucleic acid encoding a protein according to any of the following (a) and (b):
(a) a protein having an amino acid sequences set forth in any of SEQ ID NOs: 3, 4, 6, 8, and 9;
(b) a protein having an amino acid sequence having an identity of 90% or more to an amino acid sequence set forth in any of SEQ ID NOs: 3, 4, 6, 8, and 9 and having transporter activity involved in sugar transportation.
(5) The transformed plant or transformed plant cell according to (1), wherein the homologous nucleic acid is a nucleic acid encoding a protein according to any of the following (a) to (c):
(a) a protein having an amino acid sequence having a match of 33% or more with the amino acid sequence set forth in SEQ ID NO: 2 and having transporter activity involved in sugar transportation;
(b) a protein comprising an amino acid sequence having a match of 35% or more with the amino acid sequence from the N-terminus to a.a. 213 in the amino acid sequence set forth in SEQ ID NO: 2 as the region except the transmembrane domain and having transporter activity involved in sugar transportation;
(c) a protein comprising an amino acid sequence having a match of 37% or more with the amino acid sequence of a.a. 33 to 213 in the amino acid sequence set forth in SEQ ID NO: 2 as the region except the low homology region and the transmembrane domain and having transporter activity involved in sugar transportation.
(6) The transformed plant or transformed plant cell according to (1), wherein the homologous nucleic acid is a nucleic acid encoding a protein according to any of the following (a) to (c):
(a) a protein having an amino acid sequence having a match of 29% or more with the amino acid sequence set forth in SEQ ID NO: 5 and having transporter activity involved in sugar transportation;
(b) a protein comprising an amino acid sequence having a match of 39% or more with the amino acid sequence of the N-terminus to a.a. 205 in the amino acid sequence set forth in SEQ ID NO: 5 as the region except the transmembrane domain and having transporter activity involved in sugar transportation;
(c) a protein comprising an amino acid sequence having a match of 40% or more with the amino acid sequence of a.a. 30 to 205 in the amino acid sequence set forth in SEQ ID NO: 5 as the region except the low homology region and the transmembrane domain and having transporter activity involved in sugar transportation.
(7) The transformed plant or transformed plant cell according to (1), wherein the homologous nucleic acid is a nucleic acid encoding a protein according to any of the following (a) to (c):
(a) a protein having an amino acid sequence having a match of 30% or more with the amino acid sequence set forth in SEQ ID NO: 7 and having transporter activity involved in sugar transportation;
(b) a protein comprising an amino acid sequence having a match of 37% or more with the amino acid sequence of the N-terminus to a.a. 195 in the amino acid sequence set forth in SEQ ID NO: 7 as the region except the transmembrane domain and having transporter activity involved in sugar transportation;
(c) a protein comprising an amino acid sequence having a match of 39%/o or more with the amino acid sequence of a.a. 18 to 195 in the amino acid sequence set forth in SEQ ID NO: 7 as the region except the low homology region and the transmembrane domain and having transporter activity involved in sugar transportation.
(8) A transformed plant or a transformed plant cell in which a nucleic acid encoding a protein having a consensus sequence comprising the following amino acid sequence: (N/S)(V/I)xxxxxFx(S/A)(1-3aa)TFxxI(V/F/M)Kx(K/R)(S/K/T)(V/T)x(D/E)(F/Y)(S/K)x(I/V/M)PY(V/I/L)x(T/A)x(L/M)(N/S)xxLW(V/T)(V/F/L)YGL(0-2aa)(V/I/F/L)xxxxxLVx(T/S)(I/V)N(A/G)xGxx(I/L)(E/H)(L/F/M/I)xY(L/I/V)x(L/I/V)(Y/F)L xx(A/S/C)(2-4aa)(S/K/N)x(R/Q)(1-2aa)(V/I/M)xxxxxxx(L/V/I)xx(F/V/L)xx(V/I/M)xx(L/I/V)(V/T)(L/F)xx(V/I)(H/D/K)(D/S/N/G)(2-3aa)(R/K)xx(I/V/L/F)(I/V/L)Gx(L/M/I)xxx(F/L)xxxMYx(S/A)Pxx(V/A)xxxV(I/V)xx(R/K)S(V/T)(E/K)(Y/F)MPF(L/F)LS(L/F)(F/V)xF(I/L/V)N(G/A/S)xxWxxY(A/S)x(F/I/V/L)(2-3aa)Dx(F/Y)(I/V)xx(P/S)Nx(L/I)Gx(L/F/I)x(G/A)x(A/T/S)QLx(L/V)Yxx(Y/F)xx(A/S)(T/S)P and having transporter activity involved in sugar transportation is introduced and/or expression of the protein is enhanced.
(9) The transformed plant or transformed plant cell according to (8), wherein the consensus sequence comprises MVDAKQVRFIIGVIGNVISFGLFAAPAKTFWRIFKKKSVEEFSYVPYVAT(V/I)MNCML WVFYGLPVVHKDSxLVSTINGVGLVIE(L/I)FYV(G/A)(V/L)YLxYCGHK(Q/K)NxR(K/R)(K/N)ILx(Y/F)LxxEV(V/I)xV(A/V)xI(V/I)L(V/I)TLF(V/A)(I/L)K(N/G)DFxKQTFVG(V/I)I CD(V/I)FNIAMY(A/G)(S/A)PSLAI(I/F)(T/K)VV(K/R)TKS(V/T)EYMPFLLSLVCFVNA(A/G)IWT(S/T)YSLIFKIDxYVLASNGIGT(F/L)LALSQLIVYFMYYKSTPK(0-1aa)(E/D)KTVKPSEVEI(PS)(A/G)T(N/E/D)RV.
(10) The transformed plant or transformed plant cell according to (8), wherein the protein having transporter activity involved in sugar transportation is an AtSWEET8 protein or a protein encoded by a homologous nucleic acid of a nucleic acid encoding the AtSWEET8 protein.
(11) The transformed plant or transformed plant cell according to (10), wherein the AtSWEET8 protein is a protein according to any of the following (a) to (c):
(a) a protein having the amino acid sequences of SEQ ID NO: 2;
(b) a protein having an amino acid sequence having an identity of 90% or more to the amino acid sequence set forth in SEQ ID NO: 2 and having transporter activity involved in sugar transportation;
(c) a protein having an amino acid sequence encoded by a polynucleotide hybridizable with all or a part of a polynucleotide having the nucleotide sequence set forth in SEQ ID NO: 1 under stringent conditions and having transporter activity involved in sugar transportation.
(12) The transformed plant or transformed plant cell according to (10), wherein the homologous nucleic acid is a nucleic acid encoding a protein according to any of the following (a) to (c):
(a) a protein having an amino acid sequence set forth in SEQ ID NO: 5 or 7;
(b) a protein having an amino acid sequence having an identity of 90% or more to an amino acid sequence set forth in SEQ ID NO: 5 or 7 and having transporter activity involved in sugar transportation;
(c) a protein having an amino acid sequence encoded by a polynucleotide hybridizable with all or a part of a polynucleotide having a nucleotide sequence set forth in any of SEQ ID NOs: 40 to 43 under stringent conditions and having transporter activity involved in sugar transportation.
(13) The transformed plant or transformed plant cell according to (10), wherein the homologous nucleic acid is a nucleic acid encoding a protein according to any of the following (a) and (b):
(a) a protein having an amino acid sequences set forth in any of SEQ ID NOs: 3, 4, 6, 8, and 9;
(b) a protein having an amino acid sequence having an identity of 90% or more to an amino acid sequence set forth in any of SEQ ID NOs: 3, 4, 6, 8, and 9 and having transporter activity involved in sugar transportation.
(14) The transformed plant or transformed plant cell according to (10), wherein the homologous nucleic acid is a nucleic acid encoding a protein according to any of the following (a) to (c):
(a) a protein having an amino acid sequence having a match of 33% or more with the amino acid sequence set forth in SEQ ID NO: 2 and having transporter activity involved in sugar transportation;
(b) a protein comprising an amino acid sequence having a match of 35% or more with the amino acid sequence from the N-terminus to a.a. 213 in the amino acid sequence set forth in SEQ ID NO: 2 as the region except the transmembrane domain and having transporter activity involved in sugar transportation;
(c) a protein comprising an amino acid sequence having a match of 37% or more with the amino acid sequence of a.a. 33 to 213 in the amino acid sequence set forth in SEQ ID NO: 2 as the region except the low homology region and the transmembrane domain and having transporter activity involved in sugar transportation.
(15) The transformed plant or transformed plant cell according to (10), wherein the homologous nucleic acid is a nucleic acid encoding a protein according to any of the following (a) to (c):
(a) a protein having an amino acid sequence having a match of 29% or more with the amino acid sequence set forth in SEQ ID NO: 5 and having transporter activity involved in sugar transportation;
(b) a protein comprising an amino acid sequence having a match of 39% or more with the amino acid sequence of the N-terminus to a.a. 205 in the amino acid sequence set forth in SEQ ID NO: 5 as the region except the transmembrane domain and having transporter activity involved in sugar transportation;
(c) a protein comprising an amino acid sequence having a match of 40% or more with the amino acid sequence of a.a. 30 to 205 in the amino acid sequence set forth in SEQ ID NO: 5 as the region except the low homology region and the transmembrane domain and having transporter activity involved in sugar transportation.
(16) The transformed plant or transformed plant cell according to (10), wherein the homologous nucleic acid is a nucleic acid encoding a protein according to any of the following (a) to (c):
(a) a protein having an amino acid sequence having a match of 30% or more with the amino acid sequence set forth in SEQ ID NO: 7 and having transporter activity involved in sugar transportation;
(b) a protein comprising an amino acid sequence having a match of 37% or more with the amino acid sequence of the N-terminus to a.a. 195 in the amino acid sequence set forth in SEQ ID NO: 7 as the region except the transmembrane domain and having transporter activity involved in sugar transportation;
(c) a protein comprising an amino acid sequence having a match of 39% or more with the amino acid sequence of a.a. 18 to 195 in the amino acid sequence set forth in SEQ ID NO: 7 as the region except the low homology region and the transmembrane domain and having transporter activity involved in sugar transportation.
(17) The transformed plant or transformed plant cell according to (1) or (8), wherein the transformed plant is a phanerogam or derived from a phanerogam.
(18) The transformed plant or transformed plant cell according to (17), wherein the phanerogam is an angiosperm.
(19) The transformed plant or transformed plant cell according to (18), wherein the angiosperm is a monocot.
(20) The transformed plant or transformed plant cell according to (19), wherein the monocot is a plant of the family Poaceae.
(21) The transformed plant or transformed plant cell according to (20), wherein the plant of the family Poaceae is a plant of the genus Oryza.
(22) The transformed plant or transformed plant cell according to (18), wherein the angiosperm is a dicot.
(23) The transformed plant or transformed plant cell according to (22), wherein the dicot is a plant of the family Brassicaceae.
(24) The transformed plant or transformed plant cell according to (23), wherein the plant of the family Brassicaceae is a plant of the genus Arabidopsis.
(25) A method for producing an exudate, comprising the steps of cultivating or culturing the transformed plant or transformed plant cell according to any of (1) to (24); and collecting an exudate from the transformed plant or transformed plant cell.
(26) The method for producing an exudate according to (25), wherein the transformed plant or transformed plant cell is cultivated or cultured under conditions at a relative humidity of 80% RH or more.
(27) The method for producing an exudate according to (25), wherein the exudate is guttation.

The description of the present application encompasses the contents described in the description and/or the drawings of JP patent application No. 2013-273130, which is the basics of the priority of the present application.

### Advantageous Effects of Invention

According to the present invention, the sugar content in the exudate derived from plants can be greatly increased. Accordingly, transformed plants according to the present invention can produce exudate having a property such as high sugar content by introducing a nucleic acid encoding a particular transporter protein involved in sugar transportation and/or enhancing expression of the protein. Also, the method for producing an exudate according to the present invention can produce an exudate with a high sugar content by using a transformed plant in which a nucleic acid encoding a particular transporter protein involved in sugar transportation is introduced and/or expression of the protein is enhanced. Furthermore, the exudate collected from the transformed plant can be used as a raw material for producing alcohol, organic acid, alkane, and terpenoids because of its high sugar content.

### BRIEF DESCRIPTION OF DRAWINGS

FIG. 1-1 is a schematic view of a phylogenetic tree made based on the amino acid sequence of the AtSWEET8 protein.

FIG. 1-2 is an extended view of a part of the phylogenetic tree shown in FIG. 1-1.

FIG. 1-3 is an extended view of a part of the phylogenetic tree shown in FIG. 1-1.

FIG. 2-1 illustrates a result of multiple alignment analysis of XP_002870717, EOA19049, XP004230255, EDQ53581, EDQ64580, EDQ72753, and XP_001759812 with the amino acid sequence set forth in SEQ ID NO: 2.

FIG. 2-2 is a diagram illustrating a result of multiple alignment analysis of XP_002870717, EOA19049, XP004230255, EDQ53581, EDQ64580, EDQ72753, and XP_001759812 with the amino acid sequence set forth in SEQ ID NO: 2 and following FIG. 2-1.

FIG. 3 illustrates a result of multiple alignment analysis of XP_002870717 and EOA19049 with the amino acid sequence set forth in SEQ ID NO: 2.

FIG. 4 is a configuration diagram schematically illustrating a physical map of the nucleic acid AtSWEET/pRI201AN prepared in Examples.

FIG. 5 is a photograph of the part producing guttation in Arabidopsis under conditions described in Examples.

FIG. 6 is a configuration diagram schematically illustrating a physical map of the nucleic acids pZH2B_GWOx_AtSWEET8, pZH2B_GWOx_AtSWEET11, and pZH2B_GWOx_AtSWEET12 prepared in Examples.

FIG. 7 is a photograph of the part producing guttation under conditions described in Examples in rice.

### DESCRIPTION OF EMBODIMENTS

The present invention will be described in detail below.

The present invention involves introduction of a nucleic acid encoding a particular transporter protein involved in sugar transportation and/or enhancement of expression of the protein. In this way, exudates with high sugar concentrations can be collected from transformed plants in which the nucleic acid is introduced into cells and/or the expression of the protein is enhanced. As used herein, the exudate refers to a liquid oozed out of tissue in plant, including, for example, root exudate, seed exudate, guttation-liquid oozed out of the hydathode. The phenomenon in which a liquid is oozed out of the hydathode is referred to as guttation. Therefore, guttation-liquid is synonymous with guttation. In particular, the transformed plant in which a nucleic acid encoding a particular transporter protein involved in sugar transportation is introduced into cells and/or the expression of the protein is enhanced can produce guttation with high sugar concentrations.

As used herein, the meaning of nucleic acid includes naturally occurring nucleic acids such as DNA and RNA, artificial nucleic acids such as peptide nucleic acid (PNA) and nucleic acid molecules in which a base, sugar, or phosphodiester moiety is chemically modified. The meaning of the nucleic acid encoding a transporter protein involved in sugar transportation includes both of the gene in the genome and the transcription product of the gene.

As used herein, the sugar refers to a substance represented by the chemical formula Cₙ(H₂O)ₘ, including polysaccharides, oligosaccharides, disaccharides, and monosaccharides, including aldehyde and ketone derivatives of polyol and derivatives and condensation products related thereto. Glucosides in which aglycone such as alcohol, phenol, saponin, or pigment is bound to reduced group of sugar are also included. The monosaccharides may be classified into triose, tetrose, hexose, or pentose based on the number of carbon atoms and they may be classified into aldose, which has an aldehyde group, ketose, which has a ketone group, or the like based on a functional group in the molecule. The sugar may be divided into D-form and L-form according to the conformation at the asymmetric carbon most apart from the aldehyde or ketone group. Specific examples of the monosaccharides include glucose, fructose, galactose, mannose, xylose, xylulose, ribose, erythrose, threose, erythrulose, glyceraldehyde, dihydroxyacetone, etc. and specific examples of the disaccharides include sucrose (saccharose), lactose, maltose, trehalose, cellobiose, etc.

The plants according to the present invention have significantly increased amounts of sugar contained in exudate such as guttation in comparison with the wild type by introducing a nucleic acid encoding a particular transporter protein involved in sugar transportation into cells and/or enhancing expression of the protein. The protein may be expressed at the all cells in the plant tissue or it may be expressed in at least a part of the cells in the plant tissue. As used herein, the meaning of the plant tissue includes the plant organs such as leaf, stem, seed, root, and flower. In the present invention, introducing a nucleic acid means significantly increasing the molecular number per cell of the nucleic acid encoding a transporter protein in comparison with the molecular number in the wild type. In the present invention, enhancing expression of a transporter protein means increasing the expression of its transcription product and/or its translation product by modifying an expression regulatory region of a nucleic acid encoding the transporter protein and/or injecting the nucleic acid itself into a cell.

### Nucleic Acid Encoding Transporter Protein Involved in Sugar Transportation

The aforementioned “nucleic acid encoding a particular transporter protein involved in sugar transportation” refers to the nucleic acid encoding the AtSWEET8 protein in Arabidopsis and homologous nucleic acids of the nucleic acid encoding the AtSWEET8 protein in plants other than Arabidopsis. Supplementary FIG. 8 in Nature (2010) 468, 527-534 discloses a phylogenetic tree of SWEETs, transporter proteins involved in sugar transportation, based on the amino acid sequences. The document discloses SWEET proteins from thale cress (Arabidopsis thaliana), SWEET proteins from rice (Oryza sativa), SWEET proteins from bur clover (Medicago trunculata), SWEET proteins from Chlamydomonas reinhardiii, SWEET proteins from Physcomitrella patens, SWEET proteins from Petunia hybrida, SWEET proteins from Caenorhabditis elegans, and SWEET proteins from mammals. According to this phylogenetic tree, it is understood that SWEETs, transporter proteins involved in sugar transportation, are classified into five clades of I to V based on the similarity of the amino acid sequence. The aforementioned AtSWEET8 protein in Arabidopsis thaliana is classified in the clade II.

Table 1 below shows corresponding GenBank ID numbers, indexes of the protein coding regions calculated from the genome data (Index in the Genome), gene names, protein names, abbreviations of the proteins, SWEET protein clade numbers, and species of the organisms of origin of SWEET proteins from Arabidopsis thaliana, SWEET proteins from Oryza sativa, and Medicago trunculata SWEET proteins and a Petunia hybrida SWEET protein among the transporter proteins SWEETs involved in sugar transportation disclosed in the document.

TABLE 1

| GenBank | GenBank | | | | Abbreviation | | |
|---|---|---|---|---|---|---|---|
| (NCBI) ID No. | (NCBI) ID No. | Index in the | | Encoded | of Encoded | SWEET | |
| #1 | #2 | Genome | Gene Name | Protein | Protein | Clade | Organism |
| NP_564140 | SWET1_ARATH | At1g21460 | AtSWEET1 | AtSWEET1 | AtSW01 | I | Arabidopsis thaliana |
| NP_566493 | SWET2_ARATH | At3g14770 | AtSWEET2 | AtSWEET2 | AtSW02 | I | Arabidopsis thaliana |
| NP_200131 | SWET3_ARATH | At5g53190 | AtSWEET3 | AtSWEET3 | AtSW03 | I | Arabidopsis thaliana |
| NP_566829 | SWET4_ARATH | At3g28007 | AtSWEET4 | AtSWEET4 | AtSW04 | II | Arabidopsis thaliana |
| NP_201091 | SWET5_ARATH | At5g62850 | AtSWEET5 | AtSWEET5 | AtSW05 | II | Arabidopsis thaliana |
| NP_176849 | SWET6_ARATH | At1g66770 | AtSWEET6 | AtSWEET6 | AtSW06 | II | Arabidopsis thaliana |
| NP_567366 | SWET7_ARATH | At4g10850 | AtSWEET7 | AtSWEET7 | AtSW07 | II | Arabidopsis thaliana |
| NP_568579 | SWET8_ARATH | At5g40260 | AtSWEET8 | AtSWEET8 | AtSW08 | II | Arabidopsis thaliana |
| NP_181439 | AAM63257 | At2g39060 | AtSWEET9 | AtSWEET9 | AtSW09 | III | Arabidopsis thaliana |
| NP_199892 | AED95992 | At5g50790 | AtSWEET10 | AtSWEET10 | AtSW10 | III | Arabidopsis thaliana |
| NP_190443 | AEE78451 | At3g48740 | AtSWEET11 | AtSWEET11 | AtSW11 | III | Arabidopsis thaliana |
| NP_197755 | AED93195 | At5g23660 | AtSWEET12 | AtSWEET12 | AtSW12 | III | Arabidopsis thaliana |
| NP_199893 | AED95993 | At5g50800 | AtSWEET13 | AtSWEET13 | AtSW13 | III | Arabidopsis thaliana |
| NP_194231 | AEE84991 | At4g25010 | AtSWEET14 | AtSWEET14 | AtSW14 | III | Arabidopsis thaliana |
| NP_196821 | AED91859 | At5g13170 | AtSWEET15 | AtSWEET15 | AtSW15 | III | Arabidopsis thaliana |
| NP_188291 | SWT16_ARATH | At3g16690 | AtSWEET16 | AtSWEET16 | AtSW16 | IV | Arabidopsis thaliana |
| NP_193327 | SWT17_ARATH | At4g15920 | AtSWEET17 | AtSWEET17 | AtSW17 | IV | Arabidopsis thaliana |
| NP_001044998 | SWT1A_ORYSJ | Os01g0881300 | OsSWEET1a | OsSWEET1a | OsSW01a | I | Oryza sativa |
| NP_001055599 | SWT1B_ORYSJ | Os05g0426000 | OsSWEET1b | OsSWEET1b | OsSW01b | I | Oryza sativa |
| NP_001043270 | SWT2A_ORYSJ | Os01g0541800 | OsSWEET2a | OsSWEET2a | OsSW02a | I | Oryza sativa |
| NP_001043983 | SWT2B_ORYSJ | Os01g0700100 | OsSWEET2b | OsSWEET2b | OsSW02b | I | Oryza sativa |
| NP_001054926 | SWT3A_ORYSJ | Os05g0214300 | OsSWEET3a | OsSWEET3a | OsSW03a | I | Oryza sativa |
| NP_001042428 | SWT3B_ORYSJ | Os01g0220700 | OsSWEET3b | OsSWEET3b | OsSW03b | I | Oryza sativa |
| NP_001046621 | SWET4_ORYSJ | Os02g0301100 | OsSWEET4 | OsSWEET4 | OsSW04 | II | Oryza sativa |
| NP_001056475 | SWET5_ORYSJ | Os05g0588500 | OsSWEET5 | OsSWEET5 | OsSW05 | II | Oryza sativa |
| NP_001043523 | SWT6A_ORYSJ | Os01g0606000 | OsSWEET6a | OsSWEET6a | OsSW06a | II | Oryza sativa |
| NP_001043522 | SWT6B_ORYSJ | Os01g0605700 | OsSWEET6b | OsSWEET6b | OsSW06b | II | Oryza sativa |
| NP_001062690 | SWT7A_ORYSJ | Os09g0254600 | OsSWEET7a | OsSWEET7a | OsSW07a | II | Oryza sativa |
| NP_001062702 | SWT7B_ORYSJ | Os09g0258700 | OsSWEET7b | OsSWEET7b | OsSW07b | II | Oryza sativa |
| SWT7C_ORYSJ | | Os12g0178500 | OsSWEET7c | OsSWEET7c | OsSW07c | II | Oryza sativa |
| NP_001062354 | — | Os08g0535200 | OsSWEET11 | OsSWEET11 | OsSW11 | III | Oryza sativa |
| NP_001050099 | — | Os03g0347500 | OsSWEET12 | OsSWEET12 | OsSW12 | III | Oryza sativa |
| SWT13_ORYSJ | — | Os12g0476200 | OsSWEET13 | OsSWEET13 | OsSW13 | III | Oryza sativa |
| NP_001067955 | — | Os11g0508600 | OsSWEET14 | OsSWEET14 | OsSW14 | III | Oryza sativa |
| NP_001046944 | — | Os02g0513100 | OsSWEET15 | OsSWEET15 | OsSW15 | III | Oryza sativa |
| NP_001050071 | SWT16_ORYSJ | Os03g0341300 | OsSWEET16 | OsSWEET16 | OsSW16 | IV | Oryza sativa |
| XP_003617528 | — | Medtr5g092600 | MtSWEET9 | MtSWEET9 | MtSW09 | III | Medicago truncatula |
| XP_003602780 | — | Medtr3g098930 | MtSWEET10a | MtSWEET10a | MtSW10a | III | Medicago truncatula |
| AFK35161 | — | — | MtSWEET10b | MtSWEET10b | MtSW10b | III | Medicago truncatula |
| CAC44123 | — | — | MtSWEET10c | MtSWEET10c | MtSW10c | III | Medicago truncatula |
| NOD3_MEDTR | — | — | NOD3 | MtSWEET15a | MtSW15a | III | Medicago truncatula |
| XP_003620983 | — | Medtr7g005690 | MtSWEET15b | MtSWEET15b | MtSW15b | III | Medicago truncatula |
| XP_003615405 | — | Medtr5g067530 | MtSWEET15c | MtSWEET15c | MtSW15c | III | Medicago truncatula |
| XP_003593107 | — | Medtr2g007890 | MtSWEET15d | MtSWEET15d | MtSW15d | III | Medicago truncatula |
| NEC1_PETHY | — | — | NEC1 | PhNEC1 | PhNEC1 | III | Petunia hybrida |

As used herein, the word AtSWEET refers to AtSWEET1, AtSWEET2, AtSWEET3, AtSWEET4, AtSWEET5, AtSWEET6, AtSWEET7, AtSWEET8, AtSWEET9, AtSWEET10, AtSWEET11, AtSWEET12, AtSWEET13, AtSWEET14, AtSWEET15, AtSWEET16, and AtSWEETT17 in Table 1 and the word OsSWEET refers to OsSWEET1a, OsSWEET1b, OsSWEET2a, OsSWEET2b, OsSWEET3a, OsSWEET3b, OsSWEET4, OsSWEET5, OsSWEET6a, OsSWEET6b, OsSWEET7a, OsSWEET7b, OsSWEET7c, OsSWEET11, OsSWEET12, OsSWEET13, OsSWEET14, OsSWEET15, and OsSWEET16 in Table 1.

The nucleotide sequence of the coding region of the nucleic acid encoding the AtSWEET8 protein and the amino acid sequence of the protein are shown in SEQ ID NOs: 1 and 2, respectively. However, “a nucleic acid encoding a particular transporter involved in sugar transportation” in the present invention is not limited to the gene specified by the nucleotide sequence and the amino acid sequence set forth in SEQ ID NOs: 1 and 2.

For example, in the present invention, the aforementioned “nucleic acids encoding a particular transporter protein involved in sugar transportation” include homologous nucleic acids of the nucleic acid encoding the AtSWEET8 protein. The meaning of the homologous nucleic acids includes both genes evolved and diverged from a common ancestor gene and genes only having similar nucleotide sequence, unlike the evolved and diverged genes. The genes evolved and diverged from a common ancestor gene include homologous genes from 2 species (ortholog) and homology genes generated by duplication in a species (paralog). The aforementioned homologous nucleic acids of the nucleic acid encoding the AtSWEET8 protein can be readily searched and identified from known databases such as GenBank based on the nucleotide sequence set forth in SEQ ID NO: 1 for the coding region of the nucleic acid encoding the AtSWEET8 protein and the amino acid sequence set forth in SEQ ID NO: 2.

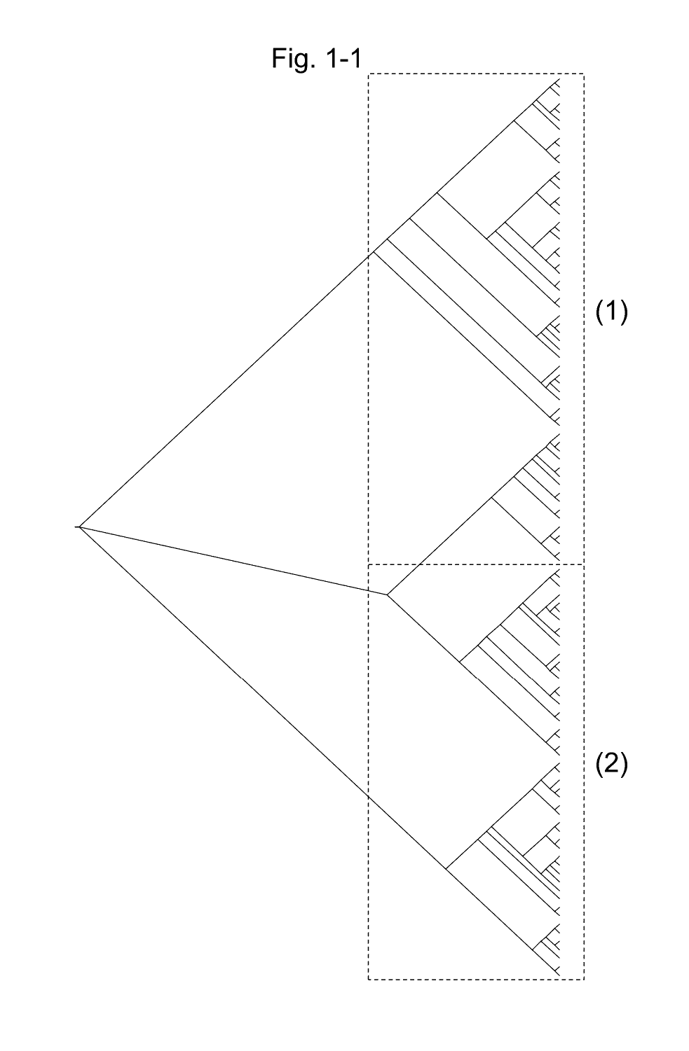

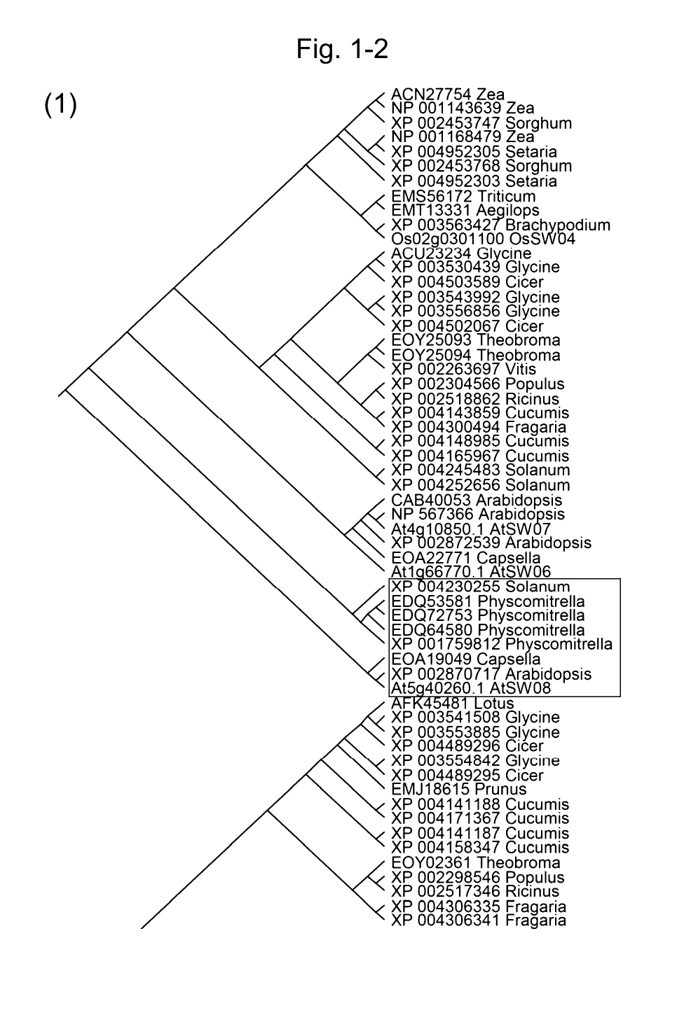

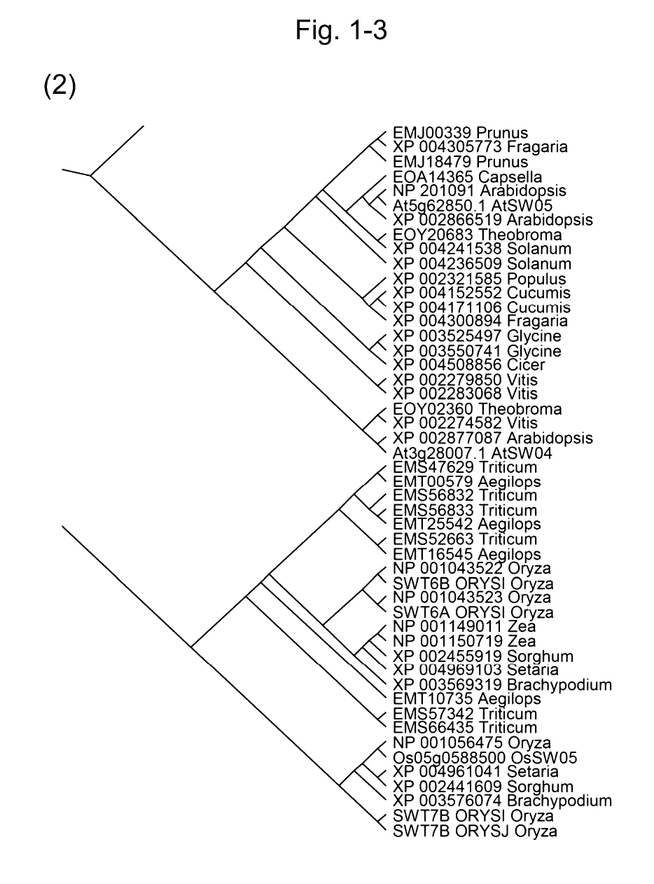

For example, the 7 nucleic acids encoding a SWEET protein from Arabidopsis thaliana (XP_002870717), a SWEET protein from Capsella rubella (EOA19049), a SWEET protein from tomato (Solanum lycopersicum) (XP004230255), 4 SWEET proteins from Physcomitrella patens (EDQ53581, EDQ64580, EDQ72753, and XP_001759812), in the box in FIG. 1-1, can be identified as the homologous nucleic acids of the nucleic acid encoding the AtSWEET8 protein from a phylogenetic tree (FIG. 1-1 to 1-3) created with ClustalW using data stored in the GenBank database. The amino acid sequence of the SWEET protein from Arabidopsis thaliana (XP_002870717) is set forth in SEQ ID NO: 3; the amino acid sequence of the SWEET protein from Capsella rubella (EOA19049) is set forth in SEQ ID NO: 4; the amino acid sequence of the SWEET protein from Solanum lycopersicum (XP004230255) is set forth in SEQ ID NO: 5; the amino acid sequence of the SWEET protein from Physcomitrella patens (EDQ53581) is set forth in SEQ ID NO: 6; the amino acid sequence of the SWEET protein from Physcomitrella patens (EDQ64580) is set forth in SEQ ID NO: 7; the amino acid sequence of the SWEET protein from Physcomitrella patens (EDQ72753) is set forth in SEQ ID NO: 8; and the amino acid sequence of the SWEET protein from Physcomitrella patens (XP_001759812) is set forth in SEQ ID NO: 9.

Moreover, examples of nucleic acids encoding the amino acid sequence (SEQ ID NO: 5) of the SWEET protein from Solanum lycopersicum (XP004230255) can include the nucleotide sequence set forth in SEQ ID NO: 40 and the nucleotide sequence set forth in SEQ ID NO: 41. Examples of nucleic acids encoding the amino acid sequence (SEQ ID NO: 7) of the SWEET protein from Physcomitrella patens (EDQ64580) can include the nucleotide sequence set forth in SEQ ID NO: 42 and the nucleotide sequence set forth in SEQ ID NO: 43.

The phylogenetic tree shown in FIG. 1-1 to 1-3 include the search result using the amino acid sequence (SEQ ID NO: 2) of the AtSWEET8 protein and the amino acid sequences of the OsSWEET4 protein, the OsSWEET5 protein, the AtSWEET4 protein, the AtSWEET5 protein, the AtSWEET6 protein, and the AtSWEET7 protein.

As described in the foregoing, examples of the “nucleic acid encoding a particular transporter protein involved in sugar transportation” can include the nucleic acids encoding the amino acid sequence set forth in SEQ ID NOs: 2 to 9 and nucleic acids including the nucleotide sequence set forth in SEQ ID NOs: 1 and 40 to 43. However, in the present invention, the aforementioned “nucleic acid encoding a particular transporter protein involved in sugar transportation” is not limited to these specific amino acid sequences and nucleotide sequences.

For example, the aforementioned “nucleic acid encoding a particular transporter protein involved in sugar transportation” may be a nucleic acid having an amino acid sequence in which one or plural amino acid sequences are deleted from, substituted with, added to, or inserted into an amino acid sequence set forth in any of SEQ ID Nos: 2 to 9 and coding a protein having transporter activity involved in sugar transportation. As used herein, the plural amino acids mean, for example, 1 to 20, preferably, 1 to 10, more preferably, 1 to 7, further preferably, 1 to 5, and most preferably, 1 to 3 amino acids. The deletion, substitution, or addition of the amino acids can be made by modifying the nucleotide sequence encoding a transporter protein involved in sugar transportation by a known technique in the art. A mutation can be introduced into a nucleotide sequence by a known technique such as the Kunkel method or the gapped duplex method or a method similar to those. For example, a mutation is introduced using a kit for introducing mutation using a site-directed mutagenesis method (using, for example, Mutant-K or Mutant-G (both trade names, TAKARA Bio Inc.) or a kit of the LA PCR in vitro Mutagenesis series (trade name, TAKARA Bio Inc.)). The method for introducing mutation may be a method involving use of a chemical mutagen as represented by EMS (ethyl methanesulfonic acid), 5-bromouracil, 2-aminopurine, hydroxylamine, N-methyl-N′-nitro-N-nitrosoguanidine, and other carcinogenic compounds, or may be a method involving radiation as represented by X-ray, alpha-ray, beta-ray, gamma-ray, and ion beam and treatment with ultraviolet.

As used herein, the nucleic acid encoding a transporter protein involved in sugar transportation means that the protein encoded by the nucleic acid has the transporter activity involved in sugar transportation. The transporter activity involved in sugar transportation is an activity measured with a FRET (Forster resonance energy transfer or fluorescence resonance energy transfer) sugar sensor localized in cytoplasm or endoplasmic reticulum (ER) for sugar transport across the ER membrane, for example, those described in Methods in Non Patent Literature 1 and 2.

Examples of the aforementioned “nucleic acid encoding a particular transporter protein involved in sugar transportation” can include genes encoding proteins having amino acid sequences having a similarity or an identity to an amino acid sequence set forth in any of SEQ ID NOs: 2 to 9 of, for example, 70% or more, preferably 80% or more, more preferably 90% or more, and most preferably 95% or more, and having transporter activity involved in sugar transportation. As used herein, the values of similarity and identity mean values calculated using a computer program equipped with a Basic Local Alignment Search Tool (BLAST) program with the default setting and a database storing genetic sequence information.

Furthermore, the aforementioned “nucleic acid encoding a particular transporter protein involved in sugar transportation” may be a nucleic acid that hybridizes under stringent conditions with all or a part of the complementary strand of a DNA having any of nucleotide sequences set forth in SEQ ID NOs: 1 and 40 to 43 and that encodes a protein having transporter activity involved in sugar transportation. As used herein, the stringent conditions refer to conditions in which so-called specific hybrids are formed, but nonspecific hybrids are not formed. For example, the stringent conditions include hybridization in 6×SSC (sodium chloride/sodium citrate) at 45° C. and then washing with 0.2 to 1×SSC, 0.1% SDS at 50 to 65° C.; or such conditions can include hybridization in 1×SSC at 65 to 70° C. and then washing with 0.3×SSC at 65 to 70° C. The hybridization can be carried out by a conventionally known method such as those described in J. Sambrook et al., Molecular Cloning. A Laboratory Manual, 2nd Ed., Cold Spring Harbor Laboratory (1989).

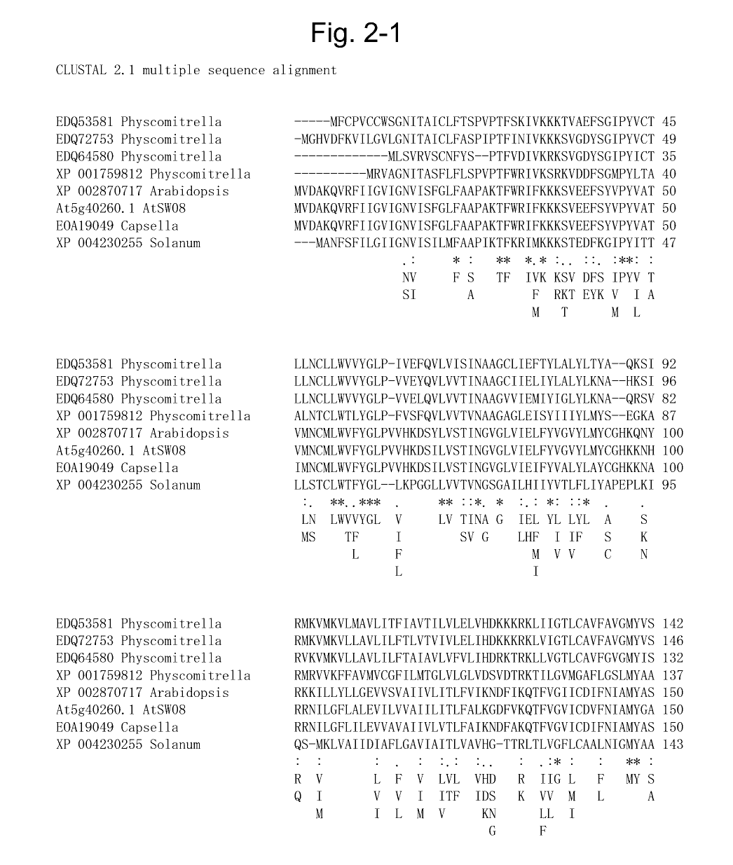

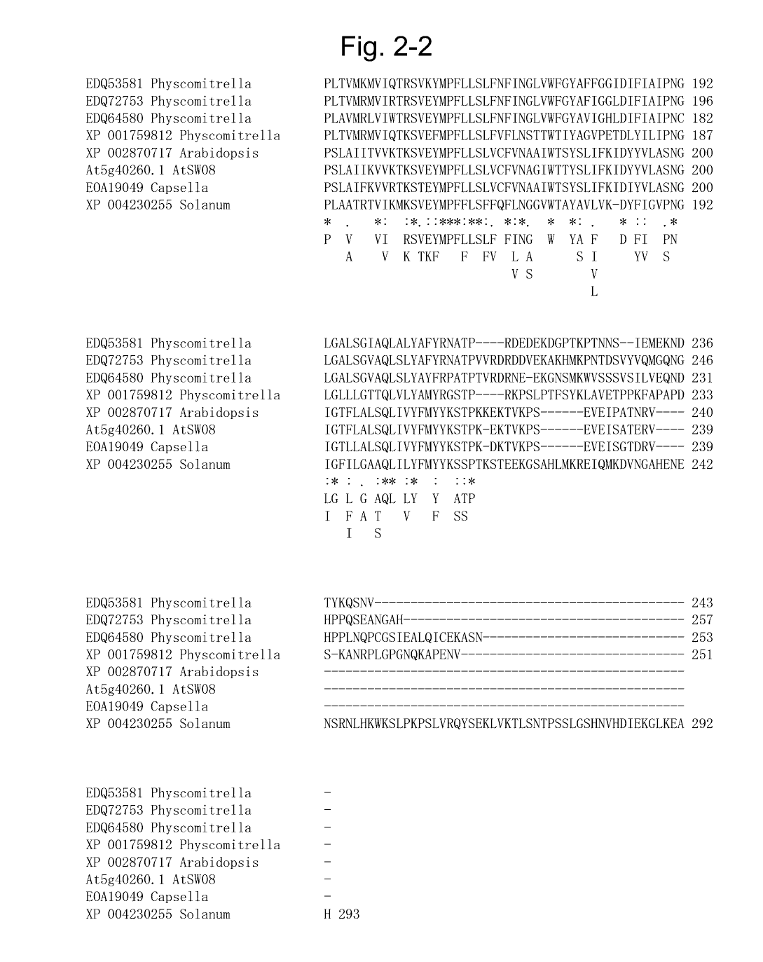

The result of a multiple alignment analysis of the SWEET protein from Arabidopsis thaliana (XP_002870717), the SWEET protein from Capsella rubella (EOA19049), the SWEET protein from Solanum lycopersicum (XP004230255), and 4 SWEET proteins from Physcomitrella patens (EDQ53581, EDQ64580, EDQ72753, and XP_001759812), shown in FIG. 1-1 to 1-3, together with the amino acid sequence set forth in SEQ ID NO: 2 is shown in FIGS. 2-1 to 2-2. As shown in FIGS. 2-1 to 2-2, the proteins encoded by the 7 homologous nucleic acids identified in the phylogenetic tree and the AtSWEET8 protein have very high matches between each other, and therefore are likely to share the function similar to that of the AtSWEET8 protein (transporter activity involved in sugar transportation) in plant.

The amino acid sequence match between the SWEET protein from Arabidopsis thaliana (XP_002870717) and the AtSWEET8 protein is 89%; the amino acid sequence match between the SWEET protein from Capsella rubella (EOA19049) and the AtSWEET8 protein is 88%; the amino acid sequence match between the SWEET protein from Solanum lycopersicum (XP004230255) and the AtSWEET8 protein is 44%; the amino acid sequence match between the SWEET protein from Physcomitrella patens (EDQ53581) and the AtSWEET8 protein is 36%; the amino acid sequence match between the SWEET protein from Physcomitrella patens (EDQ64580) and the AtSWEET8 protein is 33%; the amino acid sequence match between the SWEET protein from Physcomitrella patens (EDQ72753) and the AtSWEET8 protein is 38%; and the amino acid sequence match between the SWEET protein from Physcomitrella patens (XP_001759812) and the AtSWEET8 protein is 34%. As seen above, the aforementioned proteins encoded by the 7 homologous nucleic acids mentioned above have identities of 33% or more with the AtSWEET8 protein.

A summary of matches between the amino acid sequences of the SWEET protein from Solanum lycopersicum (XP004230255, SEQ ID NO: 5) and the SWEET protein from Physcomitrella patens (EDQ64580, SEQ ID NO: 7) and the amino acid sequences of the AtSWEET8 protein and the proteins encoded by the 6 other homologous nucleic acids is also shown in Table 2.

TABLE 2

| | numerator | | | | | | | |
|---|---|---|---|---|---|---|---|---|
| | | EDQ53581 | EDQ64580 | EDQ72753 | XP_001759812 | | | |
| | | (Physco- | (Physco- | (Physco- | (Physco- | XP_004230255 | EOA19049 | XP_002870717 |
| | | mitrella | mitrella | mitrella | mitrella | (Solanum | (Capsella | (Arabidopsis |
| denominator | AtSWEET8 | patens) | patens) | patens) | patens) | lycopersicum) | rubella) | lyrata) |
| AtSWEET8 | | 36% | 33% | 38% | 34% | 44% | 88% | 89% |
| 1aa-239aa | | | | | | | | |
| XP_004230255 | 36% | 29% | 33% | 35% | 34% | | 33% | 34% |
| (Solanum | | | | | | | | |
| lycopersicum) | | | | | | | | |
| 1aa-293aa | | | | | | | | |
| EDQ64580 | 31% | 61% | | 70% | 40% | 38% | 30% | 31% |
| (Physcomitrella | | | | | | | | |
| patens) | | | | | | | | |
| 1aa-253aa | | | | | | | | |

As shown in Table 2, the SWEET protein from Solanum lycopersicum (XP004230255) has identities of 29% or more with the AtSWEET8 protein and the proteins encoded by the other 6 homologous nucleic acids. Also, as shown in Table 2, the SWEET protein from Physcomitrella patens (EDQ64580) has identities of 30% or more with the AtSWEET8 protein and the proteins encoded by the other 6 homologous nucleic acids.

Judging from the result shown in Table 2, the “nucleic acid encoding a particular transporter protein involved in sugar transportation” may be a nucleic acid encoding a protein having an amino acid sequence having a match of 33% or more with the amino acid sequence set forth in SEQ ID NO: 2, an amino acid sequence having a match of 29% or more with the amino acid sequence set forth in SEQ ID NO: 5, or an amino acid sequence having a match of 30% or more with the amino acid sequence set forth in SEQ ID NO: 7, and having transporter activity involved in sugar transportation.

The AtSWEET8 protein and the aforementioned proteins encoded by the 7 homologous nucleic acids have a transmembrane domain in the C-terminal side. The transmembrane domain in the AtSWEET8 protein is from a.a. 214 to the C-terminus in the amino acid sequence set forth in SEQ ID NO: 2. The transmembrane domain in the SWEET protein from Solanum lycopersicum (XP004230255) is from a.a. 206 to the C-terminus in the amino acid sequence set forth in SEQ ID NO: 5. The transmembrane domain in the SWEET protein from Physcomitrella patens (EDQ64580) is from a.a. 196 to the C-terminus in the amino acid sequence set forth in SEQ ID NO: 7.

A summary of matches between the amino acid sequences of the region except the transmembrane domain in the AtSWEET8 protein, the SWEET protein from Solanum lycopersicum (XP004230255), and the SWEET protein from Physcomitrella patens (EDQ64580) and the amino acid sequences of the AtSWEET8 protein and the proteins encoded by the other 6 homologous nucleic acids is shown in Table 3.

TABLE 3

| | numerator | | | | | | | |
|---|---|---|---|---|---|---|---|---|
| | | EDQ53581 | EDQ64580 | EDQ72753 | XP_001759812 | | | |
| | | (Physco- | (Physco- | (Physco- | (Physco- | XP_004230255 | EOA19049 | XP_002870717 |
| | | mitrella | mitrella | mitrella | mitrella | (Solanum | (Capsella | (Arabidopsis |
| denominator | AtSWEET8 | patens) | patens) | patens) | patens) | lycopersicum) | rubella) | lyrata) |
| AtSWEET8 | | 36% | 35% | 39% | 36% | 43% | 88% | 88% |
| 1aa-213aa | | | | | | | | |
| XP_004230255 | 45% | 39% | 40% | 46% | 41% | | 42% | 43% |
| (Solanum | | | | | | | | |
| lycopersicum) | | | | | | | | |
| 1aa-205aa | | | | | | | | |
| EDQ64580 | 38% | 70% | | 80% | 47% | 43% | 37% | 38% |
| (Physcomitrella | | | | | | | | |
| patens) | | | | | | | | |
| 1aa-195aa | | | | | | | | |

As shown in Table 3, the region except the transmembrane domain in the AtSWEET8 protein has a match of 35% or more with the regions except the transmembrane domains in the aforementioned proteins encoded by the 7 homologous nucleic acids. Also, as shown in Table 3, the region except the transmembrane domain in the SWEET protein from Solanum lycopersicum (XP004230255) has an identity of 39% or more with the region except the transmembrane domain in the AtSWEET8 protein and the regions except the transmembrane domains in the proteins encoded by the other 6 homologous nucleic acids. Furthermore, as shown in Table 3, the region except the transmembrane domain in the SWEET protein from Physcomitrella patens (EDQ64580) has an identity of 37% or more with the region except the transmembrane domain in the AtSWEET8 protein and the regions except the transmembrane domains in the proteins encoded by the other 6 homologous nucleic acids.

Judging from the result shown in Table 3, the “nucleic acid encoding a particular transporter protein involved in sugar transportation” may be a nucleic acid encoding a protein containing an amino acid sequence having an identity of 35% or more with the amino acid sequence from the N-terminus to a.a. 213 in the amino acid sequence set forth in SEQ ID NO: 2; an amino acid sequence having an identity of 39% or more with the amino acid sequence from the N-terminus to a.a. 205 in the amino acid sequence set forth in SEQ ID NO: 5; or an amino acid sequence having an identity of 37% or more with the amino acid sequence from the N-terminus to a.a. 195 in the amino acid sequence set forth in SEQ ID NO: 7 as the region except the transmembrane domain, and having transporter activity involved in sugar transportation.

The AtSWEET8 protein and the aforementioned proteins encoded by the 7 homologous nucleic acids have a low homology region in the N-terminal side. The low homology region in the AtSWEET8 protein is from the N-terminus to a.a. 32 in the amino acid sequence set forth in SEQ ID NO: 2. The low homology region in the SWEET protein from Solanum lycopersicum (XP004230255) is from the N-terminus to a.a. 29 in the amino acid sequence set forth in SEQ ID NO: 5. The low homology region in the SWEET protein from Physcomitrella patens (EDQ64580) is from the N-terminus to a.a. 17 in the amino acid sequence set forth in SEQ ID NO: 7.

A summary of matches between the amino acid sequences of the region except the low homology domain and the transmembrane domain in the AtSWEET8 protein, the SWEET protein from Solanum lycopersicum (XP004230255), and the SWEET protein from Physcomitrella patens (EDQ64580) and the amino acid sequences of the AtSWEET8 protein and the proteins encoded by the other 6 homologous nucleic acids is shown in Table 4.

TABLE 4

| | numerator | | | | | | | |
|---|---|---|---|---|---|---|---|---|
| | | EDQ53581 | EDQ64580 | EDQ72753 | XP_001759812 | | | |
| | | (Physco- | (Physco- | (Physco- | (Physco- | XP_004230255 | EOA19049 | XP_002870717 |
| | | mitrella | mitrella | mitrella | mitrella | (Solanum | (Capsella | (Arabidopsis |
| denominator | AtSWEET8 | patens) | patens) | patens) | patens) | lycopersicum) | rubella) | lyrata) |
| AtSWEET8 | | 39% | 39% | 40% | 37% | 41% | 86% | 86% |
| 33aa-213aa | | | | | | | | |
| XP_004230255 | 43% | 41% | 45% | 46% | 44% | | 40% | 40% |
| (Solanum | | | | | | | | |
| lycopersicum) | | | | | | | | |
| 30aa-205aa | | | | | | | | |
| EDQ64580 | 40% | 73% | | 83% | 48% | 45% | 39% | 40% |
| (Physcomitrella | | | | | | | | |
| patens) | | | | | | | | |
| 18aa-195aa | | | | | | | | |

As shown in Table 4, the region except the low homology region and the transmembrane domain in the AtSWEET8 protein has matches of 37% or more with the regions except the transmembrane domain in the aforementioned proteins encoded by the 7 homologous nucleic acids. Also, as shown in Table 4, the region except the low homology region and the transmembrane domain in the SWEET protein from Solanum lycopersicum (XP004230255) has matches of 40% or more with the region except the low homology region and the transmembrane domain in the AtSWEET8 protein and the region except the low homology region and the transmembrane domain in the proteins encoded by the other 6 homologous nucleic acids. Furthermore, as shown in Table 4, the region except the low homology region and the transmembrane domain in the SWEET protein from Physcomitrella patens (EDQ64580) has matches of 39% or more with the region except the low homology region and the transmembrane domain in the AtSWEET8 protein and the region except the low homology region and the transmembrane domain in the proteins encoded by the other 6 homologous nucleic acids.

Judging from the result shown in Table 4, the “nucleic acid encoding a particular transporter protein involved in sugar transportation” may be a nucleic acid encoding a protein containing an amino acid sequence having a match of 37% or more with the amino acid sequence of a.a. 33 to 213 in the amino acid sequence set forth in SEQ ID NO: 2; an amino acid sequence having a match of 40% or more with the amino acid sequence of a.a. 30 to 205 in the amino acid sequence set forth in SEQ ID NO: 5; or an amino acid sequence having a match of 39% or more with the amino acid sequence of a.a. 18 to 195 in the amino acid sequence set forth in SEQ ID NO: 7 as the region except the low homology region and the transmembrane domain and having transporter activity involved in sugar transportation.

Based on the result of multiple alignment analysis shown in FIG. 2-1 to 2-2, the following amino acid sequence has been found as Consensus Sequence 1 of the proteins encoded by the 7 homologous nucleic acids and the AtSWEET8 protein. Accordingly, the following amino acid sequence: (N/S)(V/I)xxxxxFx(S/A)(1-3aa)TFxxI(V/F/M)Kx(K/R)(S/K/T)(V/T)x(D/E)(F/Y)(S/K)x(I/V/M)PY(V/I/L)x(T/A)x(LM)(N/S)xxLW(V/T)(V/F/L)YGL(0-2aa)(V/I/F/L)xxxxxLVx(T/S)(I/V)N(A/G)xGxx(I/L)(E/H)(L/F/M/I)xY(L/I/V)x(L/I/V)(Y/F)L xx(A/S/C)(2-4aa)(S/K/N)x(R/Q)(1-2aa)(V/I/M)xxxxxxx(L/V/I)xx(F/V/L)xx(V/I/M)xx(L/I/V)(V/T)(L/F)xx(V/I)(H/D/K)(D/S/N/G)(2-3aa)(R/K)xx(I/V/L/F)(I/V/L)Gx(L/M/I)xxx(F/L)xxxMYx(S/A)Pxx(V/A)xxxV(I/V)xx(R/K)S(V/T)(E/K)(Y/F)MPF(L/F)LS(L/F)(F/V)xF(I/L/V)N(G/A/S)xxWxxY(A/S)x(F/I/V/L)(2-3aa)Dx(F/Y)(I/V)xx(P/S)Nx(L/I)Gx(L/F/I)x(G/A)x(A/T/S)QLx(L/V)Yxx(Y/F)xx(A/S)(T/S)P from the N-terminus to the C-terminus is Consensus Sequence 1.

In the amino acid sequence shown above, x denotes an arbitrary amino acid residue. In the amino acid sequence, the notations with 2 numbers connected by—and the following “aa” indicate that there is a sequence of arbitrary amino acids at the position and that the sequence consists of a number of amino acid residues, where the number is in the range between the 2 numbers. In the amino acid sequence, the notations with plural amino acids separated by/in a parenthesis indicate that there is one of the plural amino acids at the position. This way of notation is adopted in the description of the amino acid sequences herein.

Consensus Sequence 1 shown above can be in other words an amino acid sequence in which the amino acid sequence set forth in SEQ ID NO: 44, 1 to 3 arbitrary amino acid residues, the amino acid sequence set forth in SEQ ID NO: 45, 0 to 2 arbitrary amino acid residues, the amino acid sequence set forth in SEQ ID NO: 46, 2 to 4 arbitrary amino acid residues, the amino acid sequence set forth in SEQ ID NO: 47, 1 to 2 arbitrary amino acid residues, the amino acid sequence set forth in SEQ ID NO: 48, 2 to 3 arbitrary amino acid residues, the amino acid sequence set forth in SEQ ID NO: 49, 2 to 3 arbitrary amino acid residues, and the amino acid sequence set forth in SEQ ID NO: 50 are connected in this order from the N-terminus to the C-terminus.

This Consensus Sequence 1 is a sequence that is characteristic in the group consisting of the proteins encoded by the 7 homologous nucleic acids shown in FIGS. 2-1 to 2-2 and the SWEET8 protein and that is a criterion for the clear distinction from other transporter proteins involved in sugar transportation.

Accordingly, in the present invention, the aforementioned “nucleic acids encoding a particular transporter protein involved in sugar transportation” include also nucleic acids encoding proteins having an amino acid sequence set forth in Consensus Sequence 1.

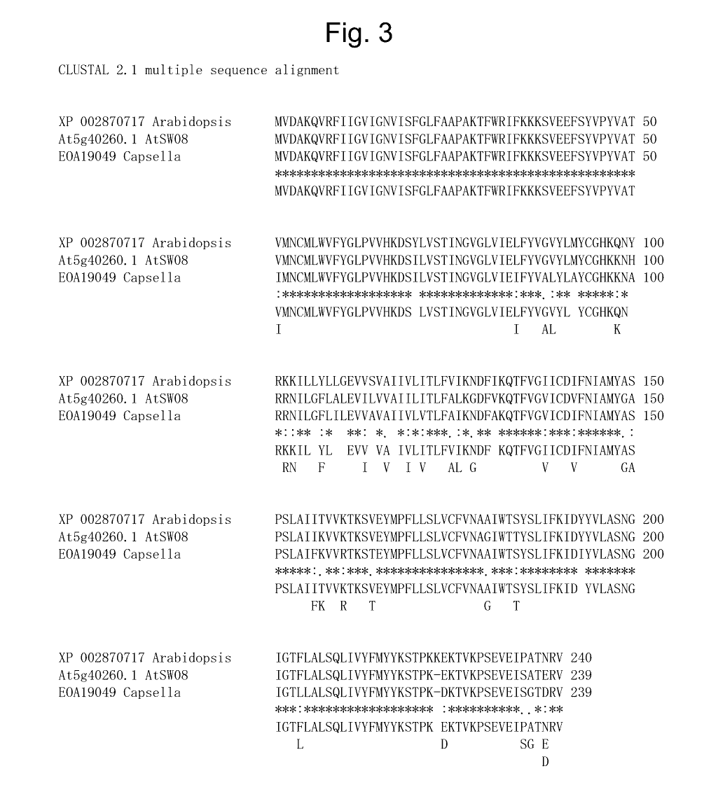

Among these, the SWEET protein from Arabidopsis thaliana (XP_002870717) and the SWEET protein from Capsella rubella (EOA19049) are found to have amino acid sequences having higher matches to the amino acid sequence of the AtSWEET8 protein as shown in FIGS. 1-1 to 1-3. A result of multiple alignment analysis of the amino acid sequences of these SWEET protein from Arabidopsis thaliana (XP_002870717) and SWEET protein from Capsella rubella (EOA19049) and the AtSWEET8 protein is shown in FIG. 3. As shown in Table 3, the SWEET protein from Arabidopsis thaliana (XP_002870717) and the SWEET protein from Capsella rubella (EOA19049) are likely to have a function (transporter activity involved in sugar transportation) similar to that of the AtSWEET8 protein in plant.

Based on the result of multiple alignment analysis shown in FIG. 3, the following amino acid sequence has been found as Consensus Sequence 2 of the SWEET protein from Arabidopsis thaliana (XP_002870717) and the SWEET protein from Capsella rubella (EOA19049) and the AtSWEET8 protein. Accordingly, the following amino acid sequence: MVDAKQVRFIIGVIGNVISFGLFAAPAKTFWRIFKKKSVEEFSYVPYVAT(V/I)MNCML WVFYGLPVVHKDSxLVSTINGVGLVIE(L/I)FYV(G/A)(V/L)YLxYCGHK(Q/K)NxR(K/R)(K/N)ILx(Y/F)LxxEV(V/I)xV(A/V)xI(V/I)L(V/I)TLF(V/A)(IL)K(N/G)DFxKQTFVG(V/I)I CD(V/I)FNIAMY(A/G)(S/A)PSLAI(I/F)(T/K)VV(K/R)TKS(V/T)EYMPFLLSLVCFVNA(A/G)IWT(S/T)YSLIFKIDxYVLASNGIGT(F/L)LALSQLIVYFMYYKSTPK(0-1aa)(E/D)KTVKPSEVEI(P/S)(A/G)T(N/E/D)RV from the N-terminus to the C-terminus is Consensus Sequence 2.

Consensus Sequence 2 shown above can be in other words an amino acid sequence in which the amino acid sequence set forth in SEQ ID NO: 51, 0 to 1 arbitrary amino acid residue, and the amino acid sequence set forth in SEQ ID NO: 52 are connected in this order from the N-terminus to the C-terminus.

This Consensus Sequence 2 is a sequence that is characteristic in the group consisting of the proteins encoded by the 2 homologous nucleic acids shown in FIG. 3 and the SWEETS protein and that is a criterion for the clear distinction from other transporter proteins involved in sugar transportation.

Accordingly, in the present invention, the aforementioned “nucleic acids encoding a particular transporter protein involved in sugar transportation” include also nucleic acids encoding proteins having an amino acid sequence set forth in Consensus Sequence 2.

The variations of amino acid residues that can occur at the certain positions in Consensus Sequence 1 shown above are based on the following reasons. It is well known that the amino acids are classified according to their side chains of similar properties (chemical properties and the physical size) as described in Reference (1) (“McKee's Biochemistry,” 3rd edition, Chapter 5 Amino acid, peptide, protein, 5.1 Amino acid, Japanese Edition supervised by Atsushi Ichikawa, translation supervised by Shinnichi Fukuoka, published by Ryosuke Sone, from Kagaku-Dojin Publishing Company, inc., ISBN4-7598-0944-9). Also, it is well known that substitution process in molecular evolution occurs frequently between amino acid residues classified in a certain group while maintaining the activity of protein. Based on this idea, a score matrix (BLOSUM) for the amino acid residue substitution is proposed in FIG. 2 in References (2): Henikoff S., Henikoff J. G., Amino-acid substitution matrices from protein blocks, Proc. Natl. Acad. Sci. USA, 89, 10915-10919 (1992) and used widely. Reference (2) is based on the findings that the substitution between amino acids having side chains of similar chemical properties has a less impact on the structure and function of the whole protein. According to References (1) and (2) mentioned above, the groups of side chains of amino acids to be considered in the multiple alignment may be those based on indexes for chemical properties, the physical size, etc. These are shown as the groups of amino acids having scores of 0 or more, or preferably amino acids having 1 or more in the score matrix (BLOSUM) disclosed in References (2). Representative groups include the following 8 groups. Another sub-grouping may be the groups of amino acids having scores of 0 or more, preferably the groups of amino acids having scores of 1 or more, or more preferably the groups of amino acids having scores of 2 or more.

### 1) Aliphatic Hydrophobic Amino Acid Group (ILMV Group)

This group is a group of the amino acids having an aliphatic hydrophobic side chain among the neutral non-polar amino acids shown in Reference (1) mentioned above and constituted of valine (V, Val), leucine (L, Leu), isoleucine (I, Ile), and methionine (M, Met). Among the amino acids classified as neutral non-polar amino acids in Reference (1), FGACWP are not included in this “aliphatic hydrophobic amino acid group” for the following reasons. Glycine (G, Gly) and alanine (A, Ala) have weak effects of the nonpolar groups because the sizes are not larger than the methyl group. Cysteine (C, Cys) may play an important role in S—S bonding and also have a property of forming hydrogen bonding with the oxygen atom and the nitrogen atom in nature. Phenylalanine (F, Phe) and tryptophan (W, Trp) have a side chain having a high molecular weight and a strong effect of the aromatic group. Proline (P, Pro) has a strong effect of the imino acid group, and fixes the angle of the main chain of polypeptide.

### 2) Group Having Hydroxy Methylene Group (ST Group)

This group is a group of amino acids having a hydroxy methylene group in the side chain among the neutral polar amino acids, and constituted of serine (S, Ser) and threonine (T, Thr). Because the hydroxyl group in the side chains of S and T is a sugar-binding site, they are often important sites for a particular activity of a certain polypeptide (protein).

### 3) Acidic Amino Acid (DE Group)

This group is a group of amino acids having an acidic carboxyl group in the side chain, and constituted of aspartic acid (D, Asp) and glutamic acid (E, Glu).

### 4) Basic Amino Acid (KR Group)

This group is a group of the basic amino acids, and constituted of lysine (K, Lys) and arginine (R, Arg). These K and R are positively charged and display basic characteristics in a wide range of pH. On the other hand, histidine (H, His), which is classified as a basic amino acid, is not classified in this group because it is hardly ionized at pH 7

### 5) Methylene Group=Polar Group (DHN Group)

In this group, all amino acids characteristically have, as a side chain, a methylene group bound to the α carbon atom and a polar group attached to the methylene group. They are characterized by having a methylene group, which is a nonpolar group, similar in physical size, and the group is constituted of asparagine (N, Asn, the polar group is the amido group), aspartic acid (D, Asp, the polar group is the carboxyl group), and histidine (H, His, the polar group is the imidazole group).

### 6) Dimethylene Group-Polar Group (EKQR Group)

In this group, all amino acids characteristically have, as a side chain, a linear hydrocarbon equal to or longer than the dimethylene group bound to the α carbon atom and a polar group attached to the hydrocarbon. They are characterized by having a dimethylene group, which is a nonpolar group, similar in physical size. The group is constituted of glutamic acid (E, Glu, the polar group is the carboxyl group), lysine (K, Lys, the polar group is the amino group), glutamine (Q, Gin, the polar group is the amido group), and arginine (R, Arg, the polar groups are the imino group and the amino group).

### 7) Aromatic (FYW Group)

This group is a group of aromatic amino acids, which have a benzene nucleus in the side chain and characterized by chemical properties unique to aromatic groups. The group consists of phenylalanine (F, Phe), tyrosine (Y, Tyr), and tryptophan (W, Trp).

### 8) Cyclic & Polar (HY Group)

This group is a group of amino acids that has a ring structure and polarity in the side chain, and constituted of histidine (H, His, the ring structure and the polar group are both the imidazole group), tyrosine (Y, Tyr, the ring structure is the benzene nucleus and the polar group is the hydroxyl group).

Based on the aforementioned amino acid groups, substitution of an amino acid residue in the amino acid sequence of a protein having a certain function with an amino acid residue in the same group can be easily expected to result in a novel protein having a similar function. For example, based on the aforementioned “1) Aliphatic hydrophobic amino acid group (ILMV group),” substitution of an isoleucine residue in the amino acid sequence of a protein having a certain function with a leucine residue can be easily expected to result in a novel protein having a similar function. If there are multiple proteins having a certain function, their amino acid sequences may be expressed as a consensus sequence. Also in such a case, substitution of an amino acid residue with an amino acid residue in the same group can be easily expected to result in a novel protein having a similar function. For example, if there are multiple proteins having a certain function and an amino acid residue in the consensus sequence calculated from them is isoleucine or leucine (L/I), based on the aforementioned “1) Aliphatic hydrophobic amino acid group (ILMV group)”, substitution of the isoleucine or leucine residue with a methionine or valine residue can be easily expected to result in a novel protein having a similar function.

The plant to which the present invention is applied can produce a high sugar concentration exudate (e.g., guttation) by introducing a nucleic acid encoding a “particular transporter protein involved in sugar transportation” as defined above into a cell, or enhancing the expression of the protein encoded by the nucleic acid. Examples of techniques for introducing the nucleic acid encoding this transporter involved in sugar transportation into a cell can include, for example, a technique for introducing into a cell an expression vector in which a DNA encoding the transporter involved in sugar transportation is placed to allow the expression thereof. Also, examples of a technique for enhancing the expression of the nucleic acid encoding the transporter involved in sugar transportation can include a technique for modifying a transcriptional promoter located in proximate to the DNA encoding the transporter involved in sugar transportation in a plant of interest. In particular, a technique for introducing in a cell in the plant of interest an expression vector in which a DNA encoding the aforementioned transporter involved in sugar transportation is placed under the control of a promoter enabling constant expression to allow the expression thereof is preferred.

### Expression Vector

The expression vector is constructed to contain a nucleic acid having a promoter nucleotide sequence that allows constitutional expression and the nucleic acid encoding the transporter involved in sugar transportation. A variety of conventionally known vectors can be used as a base vector from which the expression vector is derived. For example, a plasmid, a bacteriophage, or a cosmid can be used and selected appropriately depending on the plant cell into which the vector is introduced and the method of introduction. Specific examples can include, for example, pBR322, pBR325, pUC19, pUC119, pBluescript, pBluescriptSK, and pBI vectors. In particular, use of a binary pBI vector is preferred when the method for introducing the vector into the plant cell is a method involving use of Agrobacterium. Specific examples of the binary pIB vector can include pBIG, pBIN19, pBI101, pBI121, pBI221, etc.

The promoter is not particularly limited, as long as it is a promoter capable of allowing the expression of the nucleic acid encoding the transporter involved in sugar transportation in the plant, and a known promoter can be preferably used. Examples of such a promoter can include, for example, cauliflower mosaic virus 35S promoter (CaMV35S), various actin gene promoters, various ubiquitin gene promoters, the nopaline synthetase gene promoter, the PR1a gene promoter in tobacco, ribulose 1 in tomato, the 5-diphosphate carboxylase/oxidase small subunit gene promoter, the napin gene promoter, the oleosin gene promoter, etc. Among these, use of cauliflower mosaic virus 35S promoter, an actin gene promoter, or a ubiquitin gene promoter can be more preferred. Use of any of the aforementioned promoter allows strong expression of any nucleic acid when introduced in a plant cell.

Promoters that can be used include promoters having the function to express a nucleic acid region specifically in plant. Such a promoter that can be used may be any promoter conventionally known. By using such a promoter and region specifically introducing the aforementioned nucleic acid encoding the transporter involved in sugar transportation, the sugar content can be increased in the exudate produced from the plant organ or tissue composed of the cells into which the nucleic acid has been introduced.

The expression vector may further comprise a nucleic acid having another segment sequence in addition to the promoter and the aforementioned nucleic acid encoding the transporter involved in sugar transportation. The nucleic acid having another segment sequence is not particularly limited and examples can include a nucleic acid having a terminator nucleotide sequence, a nucleic acid having a transformant selection marker nucleotide sequence, a nucleic acid having an enhancer nucleotide sequence, a nucleic acid having a nucleotide sequence for increasing the translation efficiency, etc. Moreover, the aforementioned recombinant expression vector may have a T-DNA region. The T-DNA region can increase the efficiency of introduction of nucleic acid, especially when introducing a nucleic acid having the aforementioned nucleotide sequence in the recombination expression vector into a plant cell using Agrobacterium.

The nucleic acid having a terminator nucleotide sequence is not particularly limited as long as it has the function as a transcription termination site, and may be a known one. Specific examples of the nucleic acid that can be used include the terminator region of nopaline synthetase gene (Nos terminator), the terminator region of cauliflower mosaic virus 35S (CaMV35S terminator), etc. In particular, use of the Nos terminator may be more preferred. In the aforementioned recombinant vector, placing a terminator at an appropriate position may prevent the synthesis of needlessly long transcript after the vector is introduced into a plant cell.

Examples of the nucleic acid having a transformant selection marker nucleotide sequence that can be used include a nucleic acid containing a drug-resistance gene. Specific examples of such a drug-resistance gene can include nucleic acids containing drug-resistance genes for hygromycin, bleomycin, kanamycin, gentamicin, chloramphenicol, etc. This allows the facilitated selection of transformed plants by selecting plants growing in media containing the aforementioned antibiotics.

Examples of the nucleic acid having a nucleotide sequence for increasing the efficiency of translation can include a nucleic acid having the omega sequence derived from tobacco mosaic virus. By placing this nucleic acid having the omega sequence in the noncoding region (5′ UTR) upstream of the protein coding region, the efficiency of expression of the aforementioned nucleic acid encoding a transporter involved in sugar transportation can be increased. As seen above, nucleic acids having various DNA segment sequences can be included in the aforementioned recombinant expression vector depending on its purpose.

Methods for constructing the recombinant expression vector are not particularly limited and the recombinant expression vector can be constructed by introducing the aforementioned nucleic acid having a promoter nucleotide sequence, the nucleic acid encoding the transporter protein involved in sugar transportation, and optionally the aforementioned nucleic acid having another DNA segment sequence into the base vector selected as appropriate in a certain order. For example, the recombinant expression vector can be constructed by ligating the nucleic acid encoding a transporter involved in sugar transportation, the nucleic acid having a promoter nucleotide sequence, and (optionally the nucleic acid having a terminator nucleotide sequence) and introducing this into the vector.

Methods for replicating (methods for producing) the aforementioned expression vector are not particularly limited and conventionally known methods can be used. Generally, Escherichia coli may be used as a host and the vector may be replicated in the host. Any preferred strain of Escherichia coli may be selected depending on the type of vector.

### Transformation

The aforementioned expression vector is introduced into a plant cell of interest by a general transformation method. Methods for introducing the expression vector into (methods for transforming) a plant cell are not particularly limited and conventionally known methods suitable for the plant cell can be used. Specific examples of such methods that can be used include methods involving use of Agrobacterium and methods involving direct introduction into plant cells. Examples of the methods involving use of Agrobacterium that can be used include the methods described in Bechtold, E., Ellis, J. and Pelletier, G. (1993) In Planta, Agrobacterium-mediated gene transfer by infiltration of adult Arabidopsis plants. C.R. Acad. Sci. Paris Sci. Vie, 316, 1194-1199, or Zyprian E, Kado Cl, Agrobacterium-mediated plant transformation by novel mini-T vectors in conjunction with a high-copy vir region helper plasmid. Plant Molecular Biology, 1990, 15 (2), 245-256.

Examples of the methods for directly introducing the expression vector into a plant cell that can be used include microinjection, electroporation, the polyethyleneglycol method, the particle gun method, protoplast fusion, the calcium phosphate method, etc.

When using one of the aforementioned methods for directly introducing the nucleic acid encoding the transporter involved in sugar transportation into a plant cell, a nucleic acid containing a transcription unit necessary for the expression of the nucleic acid encoding the transporter of interest, for example, a nucleic acid having a promoter nucleotide sequence and/or a nucleic acid having a transcription terminator nucleotide sequence; and the nucleic acid encoding the transporter of interest is sufficient and the vector function is not required. Furthermore, even a nucleic acid containing no transcription unit but only the protein-coding region of the aforementioned nucleic acid encoding the transporter involved in sugar transportation is sufficient, if the nucleic acid can be integrated in a transcription unit in the host genome and express the gene of interest. Also, even when the nucleic acid is not integrated in the host genome, it is sufficient if the aforementioned nucleic acid encoding the transporter involved in sugar transportation is transcribed and/or translated in the cell.

Examples of the plant cell into which the aforementioned expression vector or a nucleic acid containing no expression vector and encoding the transporter involved in sugar transportation of interest is introduced can include cells in tissues in plant organs such as flower, leaf, and root, callus, cells in suspension culture, etc. The expression vector may be an appropriate expression vector constructed for the type of plant to be produced if necessary or a preconstructed general-purpose expression vector may be introduced into a plant cell.

The plant constituted of cells into which the expression vector is introduced is not particularly limited. This means that the concentration of sugar contained in an exudate such as guttation can be increased in any plant by introducing the aforementioned nucleic acid encoding the transporter involved in sugar transportation. Preferred examples of such a plant are phanerogam plants. Among the phanerogam plants, angiosperm plants are more preferred. Examples of such angiosperm plants include, but are not limited to, dicot and monocot plants, for example, Brassicaceae, Gramineae, Solanaceae, Leguminosae, and Salicaceae plants (see below)

Brassicaceae thale cress (Arabidopsis thaliana), Arabidopsis lyrata, rape (Brassica rapa, Brassica napus, Brassica campestris), cabbage (Brassica oleracea var. capitata), Chinese cabbage (Brassica rapa var. pekinensis), napa cabbage (Brassica rapa var. chinensis), turnip (Brassica rapa var. rapa), nozawana (Brassica rapa var. hakabura), potherb mustard (Brassica rapa var. lancinifolia), komatsuna (Brassica rapa var. peruviridis), bok choy (Brassica rapa var. chinensis), komatsuna (Raphanus sativus), wasabi (Wasabia japonica), Capsella rubella, etc.

Chenopodiaceae: sugar beet (Beta vulgaris).
Aceraceae sugar maple (Acer saccharum):
Euphorbiaceae: castorbean (Ricinus communis):
Solanaceae: Tobacco (Nicotiana tabacum), eggplant (Solanum melongena), potato (Solanum tuberosum), tomato (Solanum lycopersicum), pepper (Capsicum annuum), petunia (Petunia hybrida), etc.
Fabaceae: Soybean (Glycine max), pea (Pisum sativum), broad beans (Vicia faba), Japanese wisteria (Wisteria floribunda), peanut (Arachis hypogaea), bird's-foot trefoil (Lotus japonicus), kidney bean (Phaseolus vulgaris), adzuki bean (Vigna angularis), acacia (Acacia), snail clover (Medicago truncatula), chick-pea (Cicer arietinum), etc.
Compositae: chrysanthemum (Chrysanthemum morifolium), sunflower (Helianthus annuus), etc.
Arecaceae: oil palm (Elaeis guineensis, Elaeis oleifera), coconut palm (Cocos nucifera), date palm (Phoenix dactylifera), wax palm (Copernicia), eyc.
Anacardiaceae: wax tree (Rhus succedanea), cashew tree (Anacardium occidentale), Chinese lacquer tree (Toxicodendron vernicifluum), mango (Mangifera indica), pistachio (Pistacia vera), etc.
Cucurbitaceae: pumpkin (Cucurbita maxima, Cucurbita moschata, Cucurbita pepo), cucumber (Cucumis sativus), Japanese snake gourd (Trichosanthes cucumeroides), calabash (Lagenaria siceraria var. gourda), etc.
Rosaceae: almond (Amygdalus communis), rose (Rosa), strawberry (Fragaria vesca), cherry tree (Prunus), apple (Malus pumila var. domestica), peach (Prunus persica), etc.
Vitaceae: grape (Vitis vinifera)
Caryophyllaceae: carnations (Dianthus caryophyllus), etc.
Salicaceae: poplar (Populus trichocarpa, Populus nigra, Populus tremula), etc.
Poaceae: corn (Zea mays), rice (Oryza sativa), barley (Hordeum vulgare), wheat (Triticum aestivum), red wild einkorn (Triticum urartu). Tausch's goatgrass (Aegilops tauschii), purple false brome (Brachypodium distachyon), bamboo (Phyllostachys), sugarcane (Saccharum officinarum), napier grass (Pennisetum pupureum), Erianthus (Erianthus ravenae), susuki grass (Miscanthus virgatum), sorghum (Sorghum bicolor) switchgrass (Panicum), etc.
Liliaceae: tulip (Tulipa), lily (Lilium), etc.

In particular, plants that produce relatively much exudate and have high productivity of sugar and starch, such as sugarcane, corn, rice, sorghum, wheat, sugar beet, and sugar maple, are preferred. This is because exudate collected from these plants can be used as raw materials for biofuel and bioplastics, as described in detail later.

While the nucleic acid encoding the transporter involved in sugar transportation that can be used in the present invention can be isolated from a variety of plants and used, as mentioned above, the nucleic acid can be selected as appropriate depending on the class of the plant and used. Thus, when the plant cell of interest is derived from a monocot plant, the nucleic acid encoding a transporter involved in sugar transportation to be introduced can be that isolated from a monocot plant. Also, when the plant cell of interest is derived from a dicot plant, the nucleic acid encoding a transporter involved in sugar transportation to be introduced can be that isolated from a dicot plant. Even when the plant cell of interest is derived from a monocot plant, a nucleic acid encoding a transporter involved in sugar transportation derived from a dicot plant may be introduced. The nucleic acid encoding the AtSWEET8 protein, which is a nucleic acid encoding a transporter involved in sugar transportation derived from Arabidopsis thaliana, a dicot plant, can markedly increase the amount of sugar included in exudate even when the plant into which the nucleic acid is introduced is a monocot plant such as Oryza sativa.

### Other Processes, Other Methods

After the aforementioned transformation process, a selection process for selecting an appropriate transformant from plants can be conducted by a conventionally known method. The method of the selection is not particularly limited. The appropriate transformant may be selected, for example, on the basis of drug resistance such as hygromycin resistance or by growing transformants, collecting exudate from the plants, measuring sugar contained in the collected exudate, and selecting the plant whose exudate has a concentration of sugar significantly increased in comparison with the wild type. The measurement of sugar contained in the collected exudate may be conducted by a qualitative method, but not a quantitative method. For example, the measurement may be conducted by a coloration method using a test paper that colors in response to sugar.

Progeny plants can be obtained according to a usual method from transformed plants obtained by the transformation process. By selecting progeny plants maintaining a trait associated with significantly increased expression of the aforementioned nucleic acid encoding a transporter involved in sugar transportation in comparison with the wild type on the basis of the amount of sugar contained in the exudate, stable plant strains whose exudate has an increased amount of sugar due to the trait strains can be created. From such transformed plants or progeny thereof, breeding materials such as plant cells, seeds, fruits, rootstocks, calluses, tubers, cuttings, and masses can be obtained to mass-produce, from such materials, stable plant strains whose exudate has an increased amount of sugar due to the aforementioned trait.

As described in the foregoing, the concentration of sugar contained in exudate can be significantly increased in comparison with the wild type plant by introducing a nucleic acid encoding the particular transporter involved in the aforementioned sugar transportation into a cell or enhancing the expression of the nucleic acid according to the present invention. The sugar components contained in the exudate are meant to include monosaccharide such as glucose, galactose, mannose, and fructose, and disaccharides such as sucrose, lactose, and maltose. Accordingly, by introducing the nucleic acid encoding the particular transporter involved in the sugar transportation into a cell or enhancing the expression of the gene present endogenously, the concentration of one or more of sugar components such as glucose, galactose, mannose, fructose, sucrose, lactose and maltose contained in exudate can be increased. In particular, the concentrations of glucose, fructose, and sucrose in exudate can be greatly increased according to the present invention.

In particular, when collecting guttation produced from the hydathode as exudate, it is preferred to cultivate the plant in which the nucleic acid encoding the particular transporter involved in the sugar transportation is introduced into a cell or the expression of the nucleic acid is enhanced under conditions that prevent transpiration of the produced guttation. Furthermore, it is more preferred to culture the plant under conditions in which the amount of guttation production is increased. For example, the transpiration of guttation can be prevented and the amount of guttation production can be increased by cultivating the plant in a closed space under conditions at a humidity of 80% RH or more or more preferably 90% RH or more.

For example, whereas the concentration of sugar contained in guttation of the wild type Arabidopsis thaliana is about 2.0 μM (the mean, monosaccharide equivalent), the sugar concentration is increased to about 2400 μM when the nucleic acid encoding the AtSWEET8 protein is introduced. Also, whereas the concentration of sugar contained in guttation of the wild type Oryza sativa is about 1.3 μM (the mean, monosaccharide equivalent), the sugar concentration in guttation can be greatly increased in a transformed Oryza sativa in which the nucleic acid encoding the AtSWEET8 protein has been introduced.

As described in the foregoing, exudate with a high sugar concentration can be collected according to the present invention. The collected exudate can be used for fermentative production of alcohol and/or organic acid. Accordingly, sugar components contained in exudate at high concentrations can be used as substrates for alcohol fermentation and organic acid fermentation. For example, when using guttation as exudate, the guttation collected from a plant in which the particular transporter protein gene involved in the sugar transportation is introduced or the expression of the gene present endogenously is enhanced can be directly used in reaction systems for alcohol fermentation and organic acid fermentation. Alternatively, the guttation collected from the plant can be used in reaction systems for alcohol fermentation and organic acid fermentation after a concentration process or a process for adding another carbon or nitrogen source.

### EXAMPLES

The present invention will be described in more detail with reference to Examples below. The technical scope of the present invention is however not limited to these Examples.

1. Construction of DNA Construct for Arabidopsis thaliana Transformation

### 1.1. Preparation of DNA Encoding AtSWEET Protein by PCR

### 1.1.1. Amplification of DNA Encoding AtSWEET Protein

The DNAs encoding the AtSWEET1, AtSWEET2, AtSWEET3, AtSWEET4, AtSWEET5, AtSWEET6, AtSWEET7, AtSWEET9, AtSWEET11, AtSWEET12, AtSWEET13, AtSWEET15 and AtSWEET17 proteins for assessment were amplified by PCR using cDNA prepared from Arabidopsis thaliana as a template. To insert the DNAs for assessment into the pRI201AN vector (Takara Bio Inc., #3264), forward primers to which SalI restriction enzyme recognition sequence is added to the 5′ end and reverse primers to which Sac I or Pst I restriction enzyme recognition sequence was added to the 3′ end were designed (Table 5).

TABLE 5

| Name of | | | |
|---|---|---|---|
| Amplified | | | SEQ |
| DNA | Name of Primer | Sequence | ID NO |
| SWEET1 | sal I-SWEET1-F26mer | 5′-TAATGTCGACATGAACATCGCTCACACTATCTTCGG-3′ | 10 |
| | sac I-SWEET1-R | 5′-TATGAGCTCTTAAACTTGAAGGTCTTGCTTTCCATTAAC-3′ | 11 |
| SWEET2 | sal I-SWEET2-F27mer | 5′-TAATGTCGACATGGATGTTTTTGCTTTCAATGCTTC-3′ | 12 |
| | sac I-SWEET2-R27mer | 5′-TATGAGCTCTCACACGTAAGAAACAATCAAAGGCTC-3′ | 13 |
| SWEET3 | sal I-SWEET3-F27mer | 5′-TAATGTCGACATGGGTGATAAACTTCGATTATCCATC-3′ | 14 |
| | sac I-SWEET3-R28mer | 5′-TATGAGCTCTTAGATCGATGAGGCATTGTTAGAATTC-3′ | 15 |
| SWEET4 | sal I-SWEET4-F31mer | 5′-TAATGTCGACATGGTTAACGCTACAGTTGCGAGAAACATTG-3′ | 16 |
| | sac I-SWEET4-R30mer | 5′-TATGAGCTCTCAAGCTGAAACTCGTTTAGCTTGTCCAC-3′ | 17 |
| SWEET5 | sal I-SWEET5-F30mer | 5′-TAATGTCGACATGACGGACCCCCACACCGCCCGGACGATC-3′ | 18 |
| | sac I-SWEET5-R31mer | 5′-TATGAGCTCTCAAGCCTGGCCAAGTTCGATTCCAGCATTC-3′ | 19 |
| SWEET6 | sal I-SWEET6-F33mer | 5′-TAATGTCGACATGGTGCATGAACAGTTGAATCTTATTCGGAAG-3′ | 20 |
| | sac I-SWEET6-R32mer | 5′-TATGAGCTCTCAAACGCCGCTAACTCTTTTGTTTAAATATG-3′ | 21 |
| SWEET7 | sal I-SWEET7-F28mer | 5′-TAATGTCGACATGGTGTTTGCACATTTGAACCTTCTTC-3′ | 22 |
| | sac I-SWEET7-R31mer | 5′-TATGAGCTCTTAAACATTGTTAGGTTCTTGGTTGGTATTC-3′ | 23 |
| SWEET9 | sal I-SWEET9-F31mer | 5′-TAATGTCGACATGTTCCTCAAGGTTCATGAAATTGCTTTTC-3′ | 24 |
| | sac I-SWEET9-R27mer | 5′-TATGAGCTCTCACTTCATTGGCCTCACCGATCCTTC-3′ | 25 |
| SWEET11 | sal I-SWEET11-F29mer | 5′-TAATGTCGACATGAGTCTCTTCAACACTGAAAACACATG-3′ | 26 |
| | sac I-SWEET11-R27mer | 5′-TATGAGCTCTCATGTAGCTGCTGCGGAAGAGGACTG-3′ | 27 |
| SWEET12 | sal I-SWEET12-F29mer | 5′-TAATGTCGACATGGCTCTCTTCGACACTCATAACACATG-3′ | 28 |
| | sac I-SWEET12-R29mer | 5′-TATGAGCTCTCAAGTAGTTGCAGCACTGTTTCTAACTC-3′ | 29 |
| SWEET13 | sal I-SWEET13-F30mer | 5′-GGAATTCCATATGGCTCTAACTAACAATTTATGGGCATTTG-3′ | 30 |
| | sac I-SWEET13-R30mer | 5′-TAATGTCGACTTAAACTTGACTTTGTTTCTGGACATCCTTG-3′ | 31 |
| SWEET15 | sal I-SWEET15-F30mer | 5′-TAATGTCGACATGGGAGTCATGATCAATCACCATTTCCTC-3′ | 32 |
| | sac I-SWEET15-R27mer | 5′-TATGAGCTCTCAAACGGTTTCAGGACGAGTAGCCTC-3′ | 33 |
| SWEET17 | sal I-SWEET17-F30mer | 5′-TAATGTCGACATGGCAGAGGCAAGTTTCTATATCGGAGT-3′ | 34 |
| | sac I-SWEET17-R29mer | 5′-TATGAGCTCTTAAGAGAGGAGAGGTTCAACACGTGATG-3′ | 35 |

And the PCR amplification was conducted using these primers and PrimeSTAR GXL DNA polymerase (TaKaRa, #R050A). The composition of the reaction solution was shown in Table 6 and the reaction conditions were shown in Table 7.

TABLE 6

| Component | (μl) | |
|---|---|---|
| Template DNA (100 ng/μl) | 1 | μl |
| 5 × Prime Star GXL buffer | 4 | μl |
| dNTP mixture (25 mM) | 1.6 | μl |
| Forward primer (10 ng/μl) | 0.4 | μl |
| Reverse primer (10 ng/μl) | 0.4 | μl |
| Prime Star GXL (1 u/μl) | 0.8 | μl |
| Sterile water | 12.6 | μl |
| Total | 20 | μl |

TABLE 7

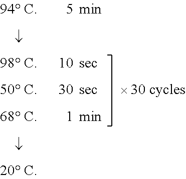

Next, the following process was conducted to add adenine to the 5′ and 3′ ends in order to insert the DNA fragments obtained by the PCR amplification into the pCR2.1-TOPO vector DNA (Invitrogen, #K4500-01). The composition of the reaction solution was shown in Table 8. The reaction solution shown in Table 8 was reacted at 70° C. for 15 minutes.

TABLE 8

| Component | | |
|---|---|---|
| PCR reaction solution | 15 | μl |
| 10 × ExTaq buffer | 3 | μl |
| dNTP mixture (25 mM) | 2 | μl |
| Ex Taq (0.5 u/μl) | 0.1 | μl |
| Sterile water | 9.9 | μl |
| Total | 30 | μl |

### 1.1.2. Cutting Out and Purification of Amplified DNA Fragment

The DNA fragments amplified by PCR were subjected to agarose gel electrophoresis and cut out and purified using MagExtractor-PCR & Gel Clean Up Kit (TOYOBO,#NPK-601). The cutting out and purification was conducted following the manual contained in the kit.

1.1.3. Transformation with Amplified DNA Fragment

The purified amplified DNA fragments were inserted into the pCR2.1-TOPO vector using TOPO TA Cloning (Invitrogen, #K4500-01). The composition of the reaction solution was shown in Table 9. The reaction solution shown in Table 9 was reacted at room temperature for 5 minutes.

TABLE 9

| Component | (μl) | |
|---|---|---|
| Cut out purification product (amplified SWEET | 2 | μl |
| sequence) | | |
| Salt solution | 0.5 | μl |
| pCR2.1-TOPO vector | 0.5 | μl |
| Total | 3 | μl |

Next, transformation was performed by adding 2 μl of this reaction solution to Escherichia coli DH5a competent cells (TOYOBO, #DNA-903). After leaving the cells in ice bath for 30 minutes, the cells were subjected to heat-treatment at 42° C. for 30 seconds. Subsequently, the cells were rapidly cooled in ice bath. 500 μl of SOC medium (Invitrogen, #15544-034) was added and the cells were cultured in suspension at 37° C., 180 rpm for 1 hour. To a LB agar plate containing kanamycin at a final concentration of 50 μg/ml, 40 mg/ml X-gal and 40 μl of 100 mM IPTG dissolved in 40 μl of DMF (N,N-dimethylformamide) were applied and then 100 to 200 μl of the culture were applied. The plate was incubated at 37° C. overnight and colonies were obtained on the next morning.

### 1.1.4. Check of Transformation by Colony PCR and Selection for Positive Clone

As a result of the transformation, many colonies were obtained. To confirm the presence or absence of the inserted DNA in the colonies, colony PCR was conducted using M13-F: 5′-GTA AAA CGA CCA GTC TTA AG-3′ (SEQ ID NO: 36) and M13-R: 5′-CAG GAA ACA GCT ATG AC-3′ (SEQ ID NO: 37). The composition of the reaction solution for the colony PCR was shown in Table 10 and the PCR conditions were shown in Table 11.

TABLE 10

| Component | (μl) | |
|---|---|---|
| DNA | Colony | |
| Amprltaq Gold 360 Master Mix (ABI, #4398881) | 10 | μl |
| Forward primer (M13-F) (10 ng/μl) | 0.4 | μl |
| Reverse primer (M13-R) (10 ng/μl) | 0.4 | μl |
| Sterile water | 9.2 | μl |
| Total | 20 | μl |

TABLE 11

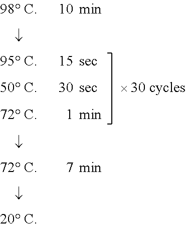

1.1.5. Purification of Plasmid DNA from Positive Clone

The plasmid DNAs were purified from the clones in which the inserted DNAs were confirmed. The purification of the plasmid DNAs were conducted using QIAprep Spin Miniprep Kit (QIAGEN, #27106) following the protocol contained in the kit.

### 1.1.6. Sequencing of Positive Clone

PCR amplification was conducted using the plasmid DNAs obtained in 1.1.5 as templates and M13-F and M13-R primers and the nucleotide sequences of the DNA fragments were determined by the dideoxy method (the Sanger method).

### 1.2. Preparation of DNA Encoding AtSWEET Protein by Chemical Synthesis

The DNA encoding the AtSWEET8, AtSWEET10, AtSWEET14, and AtSWEET16 proteins were chemically synthesized in total with their nucleotide sequences designed so as to add Pst I restriction enzyme recognition sequence to the 5′ end and Sal I restriction enzyme recognition sequence to the 3′ end. As a result, the DNAs encoding the AtSWEET8 and AtSWEET14 proteins inserted in the pEX-A vector (Operon Biotechnologies, Inc.) and the DNAs encoding the AtSWEET10 and AtSWEET16 proteins inserted in the pCR2.1-TOPO vector were able to be obtained.

### 1.3. Cutting Out of DNA Encoding AtSWEET Protein by Restriction Enzyme Reaction and Purification

In order to cut out the DNA fragments encoding the AtSWEET proteins from the plasmid DNAs obtained in 1.1.5 and 1.2, twice of restriction enzyme treatments were conducted. The combination of restriction enzymes for each DNA is shown in Table 12.

TABLE 12

| Name of DNA | First | Second |
|---|---|---|
| AtSWEET1 | Sac I | Sal I |
| AtSWEET2 | Sac I | Sal I |
| AtSWEET3 | Sac I | Sal I |
| AtSWEET4 | Sac I | Sal I |
| AtSWEET5 | Sac I | Sal I |
| AtSWEET6 | Sac I | Sal I |
| AtSWEET7 | Sac I | Sal I |
| AtSWEET8 | Nde I | Sal I |
| AtSWEET9 | Sac I | Sal I |
| AtSWEET10 | Sal I | Sac I |
| AtSWEET11 | Sac I | Sal I |
| AtSWEET12 | Sac I | Sal I |
| AtSWEET13 | Nde I | Sal I |
| AtSWEET14 | Nde I | Sal I |
| AtSWEET15 | Sac I | Sal I |
| AtSWEET16 | Sal I | Xba I |
| AtSWEET17 | Sac I | Sal I |

### 1.3.1. Sac I, Nde I, or Sal I Restriction Enzyme Reaction of Amplified DNA Fragment (First Round)

The reaction solutions shown in the tables below were prepared with Sac I (TaKaRa, #1078A), Nde I (TaKaRa, #1161A) or Sal I (TaKaRa, #1080A) and reacted at 37° C. overnight to digest the plasmids obtained in 1.1.5 or 1.2. The composition of the reaction solution with Sal I was shown in Table 13, the composition of the reaction solution with Nde I was shown in Table 14, and the composition of the reaction solution with Sac I was shown in Table 15.

TABLE 13

| Component | (μl) | |
|---|---|---|
| Plasmid | 45 | μl |
| 10 × L buffer | 10 | μl |
| Sac I | 1 | μl |
| DW | 44 | μl |
| Total | 100 | μl |

TABLE 14

| Component | (μl) | |
|---|---|---|
| Plasmid | 45 | μl |
| 10 × H buffer | 10 | μl |
| Nde I | 1 | μl |
| DW | 44 | μl |
| Total | 100 | μl |

TABLE 15

| Component | (μl) | |
|---|---|---|
| Plasmid | 45 | μl |
| 10 × H buffer | 10 | μl |
| Sal I | 1 | μl |
| DW | 44 | μl |
| Total | 100 | μl |

### 1.3.2. Purification of DNA Fragment Digested in Restriction Enzyme Reaction

Next, PCI (Phenol:Chloroform:Isoamyl alcohol=24:24:1) extraction and ethanol precipitation were performed to purify DNA. An equal volume of PCI was added to the reaction solution and the mixture was stirred and centrifuge at 15000 rpm for 5 minutes. The upper layer was collected and an equal volume of chloroform was added thereto. The mixture was similarly centrifuged and the upper layer was collected. To the collected upper layer, two times volume of ethanol was added and ethanol precipitation was conducted with Pellet Paint NF Co-Precipitant (Merck, #70748). The resultant DNA was dried and then dissolved in 44 μl of sterile water.

### 1.3.3. Sal I, Xba I, and Sac I Restriction Enzyme Reaction of Amplified DNA Fragment (Second Round)

Next, the reaction solutions shown in the tables below were prepared with Sal I (TaKaRa, #1080A), Xba I (TaKaRa, #1093A), or Sac I (TaKaRa, #1078A) and reacted at 37° C. overnight to digest the plasmids obtained in 1.3.2. The composition of the reaction solution with Sal I was shown in Table 16, the composition of the reaction solution with Xba I was shown in Table 17, and the composition of the reaction solution with Sac I was shown in Table 18.

TABLE 16

| Component | | |
|---|---|---|
| Pellet | (μl) | |
| 10 × H buffer | 5 | μl |
| Sal I | 1 | μl |
| DW | 44 | μl |
| Total | 50 | μl |

TABLE 17

| Component | | |
|---|---|---|
| Pellet | (μl) | |
| 10 × M buffer | 5 | μl |
| 100 × BSA | 0.5 | μl |
| Xba I | 1 | μl |
| DW | 43.5 | μl |
| Total | 50 | μl |

TABLE 18

| Component | | |
|---|---|---|
| Pellet | (μl) | |
| 10 × L buffer | 5 | μl |
| Sac I | 1 | μl |
| DW | 44 | μl |
| Total | 50 | μl |

### 1.3.4. Purification of DNA Fragment Digested in Restriction Enzyme Reaction

The reaction solutions obtained in 1.3.3 were subjected to agarose gel electrophoresis in a similar way to the procedure of 1.1.2 and the DNAs were cut out and purified with the MagExtractor-PCR & Gel Clean up kit.

1.4. Cutting Out of pRI201AN Vector in Restriction Enzyme Reaction and Purification

To ligate the pRI201AN vector with the DNA fragments encoding the AtSWEET proteins obtained in 1.3, the vector was treated with restriction enzymes in a way similar to the procedure of 1.3.

### 1.5. Ligation

### 1.5.1. Ligation Reaction

Ligation reaction was conducted to insert the DNA fragments encoding the AtSWEET proteins obtained in 1.3 into the pRI201AN vector obtained in 1.4. Ligation reaction was conducted with DNA Ligation Kit Ver. 2.1 (Takara Bio, #6022) at 16° C. overnight.

1.5.2. Transformation with Ligation Reaction Product

After the abovementioned ligation reaction, transformation with 2 μl of the ligation reaction solution was conducted in a way similar to 1.1.3.

### 1.5.3. Check of Ligation Reaction by Colony PCR

Insertion of the DNAs encoding the AtSWEET proteins into the vector was confirmed by examining the length of visualized DNA fragments amplified by colony PCR in agarose gel electrophoresis.

### 1.5.4. Preparation of DNA Constructs Obtained by Ligation Reaction

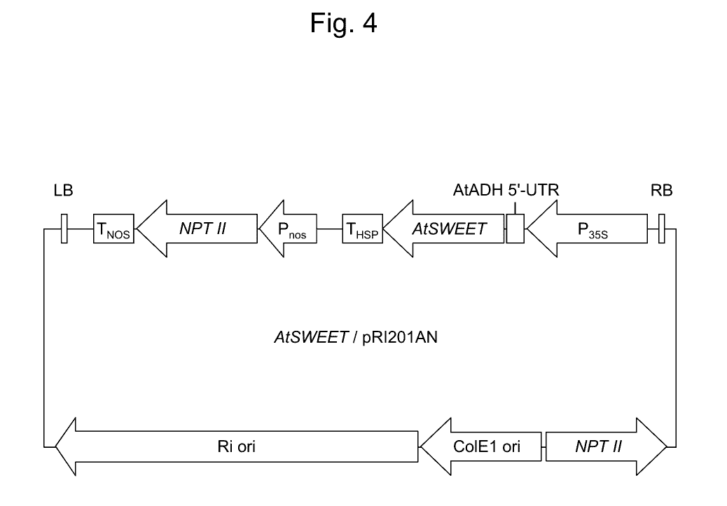

From the colonies in which the inserted DNAs were confirmed, the plasmid DNAs were purified to obtain the clones in which the DNA fragments of interest were inserted. The plasmid DNAs were purified with QIAprep Spin Miniprep Kit (QIAGEN, #27106) following the protocol contained in the kit. FIG. 4 illustrates the physical map of the resultant DNA construct (AtSWEET/pRI201AN). In FIG. 4, LB stands for left border, RB stands for right border, TNOS stands for transcription terminator of the nopaline synthetase gene NOS derived from the Ti plasmid in Agrobacterium tumefaciens, NPTII stands for neomycin phosphotransferase II gene from Escherichia coli, Pnos stands for transcription promoter of the nopaline synthetase gene NOS derived from the Ti plasmid in Agrobacterium tumefaciens. THSP stands for transcription terminator of the heat shock protein gene HSP derived from Arabidopsis thaliana, AtSWEET stands for DNA encoding a SWEET protein derived from Arabidopsis thaliana, P35S stands for Cauliflower mosaic virus 35S transcription promoter, AtADH 5′-UTR stands for translation enhancer of the alcohol dehydrogenase gene ADH derived from Arabidopsis thaliana, ColE1 ori stands for the reproduction origin of Escherichia coli, Ri ori stands for the reproduction origin of Agrobacterium rhizogenes, respectively.

### 1.6.1. Preparation of DNA Encoding OsSWEET Protein by Chemical Synthesis and Construction of Construct

The DNAs encoding the OsSWEET5, OsSWEET11, OsSWEET12, OsSWEET13, OsSWEET14, and OsSWEET15 proteins, whose nucleotide sequences were newly designed in reference to the codon usage in Arabidopsis thaliana so that there will be no change in the amino acid sequence, were designed to have an Nde I restriction enzyme recognition sequence at the start codon side and a Sac I restriction enzyme recognition sequence at the stop codon side. The designed DNAs were totally chemically synthesized and inserted into the pRI201 AN vector to obtain the respective DNA constructs. The DNAs were designed so that the ATG in the Nde I restriction enzyme recognition sequence (5′CATATG3′) added to the 5′ end coincides with the start codons of the DNAs encoding the SWEET proteins.

### 1.6.2. Preparation of DNAs Encoding SlSWEET8 and PpSWEET8 by Chemical Synthesis and Construction of Construct

SEQ ID NO: 40 was designed as a nucleotide sequence encoding the amino acid sequence of the SWEET protein derived from tomato (XP004230255, hereinafter referred to as SlSWEET8) set forth in SEQ ID NO: 5 and SEQ ID NO: 42 was designed as a nucleotide sequence encoding the SWEET protein from Physcomitrella patens set forth in SEQ ID NO: 7 (EDQ64580, hereinafter referred to as PpSWEET8). DNAs were designed so that each of them has an Nde I restriction enzyme recognition sequence at the start codon side and a Sac I restriction enzyme recognition sequence at the stop codon side of SEQ ID NO: 40 or 42. The designed DNAs were totally chemically synthesized and inserted into the pRI201AN vector to obtain the two DNA constructs. The DNAs were designed so that the ATG in the Nde I restriction enzyme recognition sequence (5′CATATG3′) added to the 5′ end coincides with the start codons in SEQ ID NOs: 40 and 42.

1.7. Transformation of Arabidopsis thaliana

The vectors for plant expression prepared in 1.5 and 1.6.1 and 1.6.2 were introduced into Agrobacterium tumefaciens strain C58C1 by electroporation (Plant Molecular Biology Mannal, Second Edition, B. G. Stanton and A. S. Robbert, Kluwer Academic Publishers 1994). Then, Agrobacterium tumefaciens in which the vectors for plant expression were each introduced was introduced into the wild type Arabidopsis thaliana ecotype Col-0 by dipping described by Clough, et al. (Steven J. Clough and Andrew F. Bent, 1998, The Plant Journal 16, 735-743) and T1 (the first generation transformant) seeds were collected. The collected T1 seeds were sown in sterile on MS agar medium (agar concentration 0.8%) containing kanamycin (50 mg/L), carbenicillin (100 mg/L) and Benlate wettable powder (10 mg/L: Sumitomo Chemical Co., Ltd.) and cultured for about 2 weeks to select transformants. The selected transformants were transplanted onto a fresh preparation of the same MS agar medium, further cultivated for about 1 week, and then transplanted in a pot containing the soil which is a 1:1 mixture of vermiculite and Soil-mix (Sakata Seed Co.) to obtain T2 (the second generation transformant) seeds.

1.8. Preparation of Arabidopsis thaliana Guttation

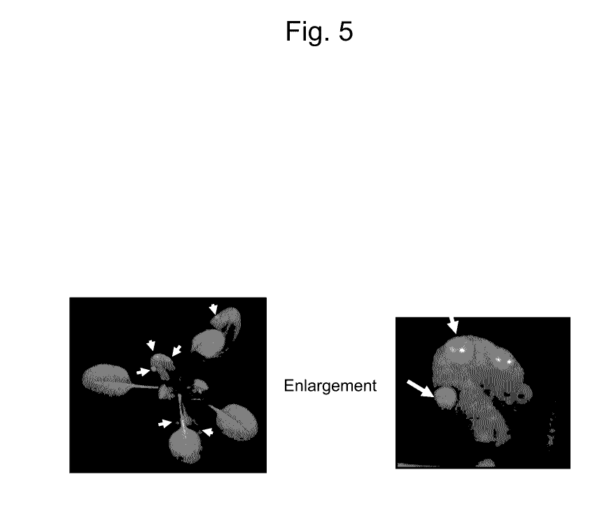

T1 or T2 plants of Arabidopsis thaliana transformed with the DNAs encoding the AtSWEET. OsSWEET, SlSWEET8, and PpSWEET8 proteins were cultivated with 18 L/6 D (24 hour cycles with 18 hours of light conditions followed by 6 hours of dark conditions) at 22° C. After acclimation, 1/1000 Hyponex was given to plants cultivated for 1 to 2 weeks and the plants were wrapped with a plastic wrap (Saran Wrap®, Asahi Chemical Industry) to increase humidity to 80% or more, or preferably 90% or more so that guttation is secreted (FIG. 5). Mainly, guttation attached to the back of leaves was collected and the sugar concentration in the guttation was analyzed. T1 seeds are defined as seeds harvested after infecting the wild type Arabidopsis thaliana with Agrobacterium and cultivating the resultant, T1 plants are defined as plants which has been confirmed to have introduction of DNA into cells, for example, by screening of T1 seeds with drug or by PCR, and T2 seeds are defined as seeds harvested after cultivating T1 plants.

2. Construction of DNA Construct for Oryza sativa Transformation

### 2.1. Amplification of DNA Encoding AtSWEET Protein

Using the aforementioned DNA constructs (the DNA encoding the AtSWEET8 protein and the DNA encoding the AtSWEET11 protein and the DNA encoding the AtSWEET12 protein) for Arabidopsis thaliana transformation prepared in 1.5.4 as templates, the DNA encoding the AtSWEET8 protein and the DNA encoding the AtSWEET11 protein and the DNA encoding the AtSWEET12 protein were amplified by PCR. The sequence CACC was added to the 5′ end of each amplification product for the introduction of the amplification product into the pENTR/D-TOPO vector.

2.2. Transformation with Amplified DNA Fragment

Parts of the resultant reaction solutions were subjected to agarose gel electrophoresis to confirm the presence of expected sizes of amplified products. The amplified products were then introduced into the pENTR/D-TOPO vector using pENTER Directional TOPO Cloning Kit (Invitrogen).

Next, Escherichia coli DH5α competent cells (Takara Bio) were transformed by adding the total amount of the reaction solutions. The cells were allowed to stand in ice bath for 30 minutes and then subjected to 45 seconds of heat treatment at 42° C. Subsequently, the cells were rapidly cooled in ice bath and 300 μl of SOC medium (Takara Bio) was added thereto. The mixture was cultured at 37° C., with shaking at 180 rpm for 1 hour and this liquid culture was plated onto an LB agar plate containing kanamycin at a final concentration of 50 μg/ml and cultured at 37° C. overnight to obtain colonies on the next morning.

### 2.3. Check of Transformation by Colony PCR and Selection for Positive Clone

Insertion of the DNAs encoding the AtSWEET proteins into the vector was confirmed by examining the length of visualized DNA fragments amplified by colony PCR in agarose gel electrophoresis.

2.4. Purification of Plasmid DNA from Positive Clone

The plasmid DNAs were purified from the clones in which the inserted DNAs were able to be confirmed. The purification of the plasmid DNAs were conducted using QIAprep Spin Miniprep Kit (QIAGEN, #27106) following the protocol contained in the kit.

### 2.5. Sequencing of Positive Clone

Using the plasmid DNAs purified in 2.4 as templates and M13-F and M13-R primers, the DNA fragments were sequenced by a DNA sequencer (Beckman Coulter, CEQ8000).

### 2.6. LR Reaction and Transformation

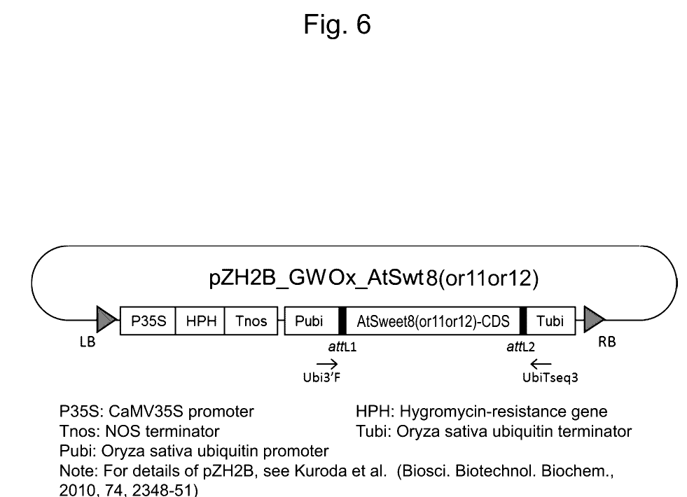

The pENTR/D-TOPO plasmid DNAs in which the DNA encoding the AtSWEET8 protein, the DNA encoding the AtSWEET11 protein, and the DNA encoding the AtSWEET12 protein were inserted obtained in 2.4 and a vector for Oryza sativa transformation (pZH2B_GWOx) were subjected to the Gateway LR reaction to construct the constructs for the overexpression in the plant of Oryza sativa, as shown in FIG. 6.

Next, Escherichia coli DH5α competent cells (Takara Bio) were transformed by adding the total amount of the reaction solutions. The cells were allowed to stand in ice bath for 30 minutes and then subjected to 45 seconds of heat treatment at 42° C. Subsequently, the cells were rapidly cooled in ice bath and 300 μl of SOC medium (Takara Bio) was added thereto. The mixture was cultured at 37° C., with shaking at 180 rpm for 1 hour. This liquid culture was plated onto an LB agar plate containing spectinomycin at a final concentration of 50 μg/ml and cultured at 37° C. overnight to obtain colonies on the next morning.

### 2.7. Check of Transformation by Colony PCR and Selection for Positive Clone

Insertion of the DNAs encoding the AtSWEET proteins into the vector was confirmed by examining the length of visualized DNA fragments amplified by colony PCR in agarose gel electrophoresis.

2.8. Purification of Plasmid DNA from Positive Clone

The plasmid DNAs were purified from the clones in which the inserted DNAs were able to be confirmed. The plasmid DNAs were purified with QIAprep Spin Miniprep Kit (QIAGEN, #27106) following the protocol contained in the kit.

### 2.9. Sequencing of Positive Clone

Using the plasmid DNAs purified in 2.8 as templates and the following primers, the DNA fragments were sequenced by the DNA sequencer (Beckman Coulter, CEQ8000).

Ubi3′F:
(SEQ ID NO: 38)
5′-TGC TGT ACT TGC TTG GTA TTG-3′
UbiTseq3:
(SEQ ID NO: 39)
5′-GGA CCA GAC CAG ACA ACC-3′

### 2.10.1. Preparation of DNA Encoding OsSWEET by Chemical Synthesis

DNAs encoding the OsSWEET13, OsSWEET14, or OsSWEET15 protein were designed to have the sequence CACC at the 5′ end for the introduction into the pENTR/D-TOPO vector. The designed DNAs were totally chemically synthesized and inserted into the pENTR/D-TOPO vector.

### 2.10.2. Preparation of DNAs Encoding SlSWEET8 and PpSWEET8 by Chemical Synthesis

Here, SEQ ID NO: 41 was designed as a nucleotide sequence encoding SlSWEET8 and SEQ ID NO: 43 was designed as a nucleotide sequence encoding PpSWEET8. DNAs were designed to have the sequence CACC at the 5′ end of SEQ ID NO: 41 or 43 for the introduction into the pENTR/D-TOPO vector. The designed DNAs were totally chemically synthesized and inserted into the pENTR/D-TOPO vector.

### 2.11. Preparation of Construct of DNA Encoding OsSWEET, SlSWEET8, or PpSWEET8 Protein

Vectors for Oryza sativa transformation were constructed using the DNAs synthesized in 2.10.1 and 2.10.2 in a way similar to 2.6 to 2.9 above.

2.12. Transformation of Oryza sativa

The DNAs encoding the AtSWEET, OsSWEET, SlSWEET8, and PpSWEET8 proteins were introduced into Oryza sativa (c.v. Nipponbare) using the aforementioned vectors for plant expression constructed in 2.9 and 2.11 according to the method described in The Plant Journal (2006) 47, 969-976.

2.13. Preparation of Oryza sativa Guttation

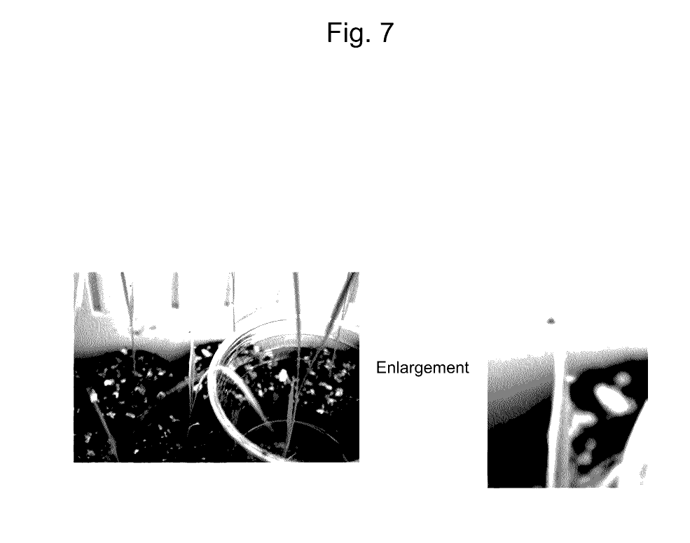

T1 transformants of Oryza sativa in which DNA encoding the AtSWEET, OsSWEET, SlSWEET8., and PpSWEET8 proteins were introduced were transplanted to a pot with a diameter of 6 cm containing 0.8 times volume of vermiculite and acclimated. Oryza sativa was cultivated with 18 L (30° C.)/6 D (25° C.) (24 hours photocycle conditions with 18 hours light conditions at 30° C. followed by 6 hours of dark conditions at 25° C.). After acclimation, 1/1000 Hyponex was sufficiently given to plants cultivated for 1 to 2 weeks and the plants were wrapped with a plastic wrap (Saran Wrap®, Asahi Chemical Industry) to increase humidity to 80% or more, or preferably 90% or more so that guttation is secreted from the hydathode in Oryza sativa (FIG. 7). Guttation attached to leaves was collected and analyzed for the sugar concentration.

### 3. Analysis for Sugar Concentration in Guttation

### 3.1. Dilution of Guttation Sample

The volumes of guttation from Arabidopsis thaliana obtained in 1.8 and guttation from Oryza sativa obtained in 2.13 were measured using a pipetter and pure water was added to a fixed volume of 0.35 ml. Next, the guttation was centrifuged at 10000×G for 10 minutes and then 0.3 mL of the supernatant was transferred to an automatic sampler vial and used for an HPLC analysis.

### 3.2. Analysis for Sugar Concentration by HPLC

The sugar concentration was analyzed using HPLC in the following conditions. In this analysis, a standard solution containing a mixture of glucose, fructose, and sucrose at 50 μM each as standard substances was used.

Analytic column: CarboPac PA1 (Dionex)

### Eluent: 100 mM NaOH

Flow rate: 1 ml/min
Amount of injection: 25 μl
Detector: Pulsed amperometric detector (Dionex ED40)

### 4. Result of Analysis

The results of measurement of sugar concentrations in guttation from Arabidopsis thaliana obtained in 1.8 and guttation from Oryza sativa obtained in 2.13 are shown in Tables 19 and 20.

TABLE 19

| | | | Glc (μM) | | | Fru (μM) | | |
|---|---|---|---|---|---|---|---|---|
| Clade | Transgene | Host | Ave | Max | Min | Ave | Max | Min |
| I | AtSW01 | A. thaliana | 1.3 | 14.2 | 0.0 | 1.8 | 11.8 | 0.0 |
| I | AtSW02 | A. thaliana | 5.7 | 33.6 | 0.0 | 0.0 | 0.0 | 0.0 |
| I | AtSW03 | A. thaliana | 4.0 | 14.7 | 0.0 | 0.9 | 6.0 | 0.0 |
| II | AtSW04 | A. thaliana | 3.0 | 9.1 | 0.0 | 8.5 | 20.7 | 0.0 |
| II | AtSW05 | A. thaliana | 5.5 | 15.7 | 0.0 | 3.4 | 20.5 | 0.0 |
| II | AtSW06 | A. thaliana | 3.3 | 10.3 | 0.0 | 0.1 | 2.0 | 0.0 |
| II | AtSW07 | A. thaliana | 4.9 | 15.1 | 0.0 | 8.0 | 19.0 | 0.0 |
| II | AtSW08 | A. thaliana | 419.9 | 838.6 | 50.2 | 610.6 | 1,154.3 | 145.6 |
| II | AtSW08 | O. sativa | 571.4 | 1,205.6 | 152.4 | 419.0 | 845.5 | 153.1 |
| III | AtSW09 | A. thaliana | 399.5 | 2,708.3 | 36.4 | 552.5 | 2,838.7 | 69.5 |
| III | AtSW10 | A. thaliana | 331.6 | 586.3 | 77.0 | 650.9 | 1,085.9 | 215.8 |
| III | AtSW11 | A. thaliana | 711.1 | 2,137.9 | 80.1 | 674.6 | 1,384.7 | 117.6 |
| III | AtSW11 | O. sativa | 31,304.7 | 59,730.0 | 757.3 | 36,772.0 | 74,830.9 | 964.0 |
| III | AtSW12 | A. thaliana | 1,375.5 | 2,920.7 | 183.4 | 1,720.7 | 3,542.4 | 201.4 |
| III | AtSW12 | O. sativa | 14,006.2 | 45,976.5 | 1,081.6 | 11,477.3 | 43,830.5 | 1,690.7 |
| III | AtSW13 | A. thaliana | 230.5 | 941.5 | 51.1 | 304.3 | 1,336.8 | 85.5 |

| | | | | Total Monosacharide | | |
|---|---|---|---|---|---|---|
| | Suc (μM) | | | Equivalent (μM) | | |
| Clade | Ave | Max | Min | Ave | Max | Min |
| I | 0.0 | 0.0 | 0.0 | 3.1 | 22.1 | 0.0 |
| I | 0.1 | 1.8 | 0.0 | 5.8 | 33.6 | 0.0 |
| I | 0.2 | 3.4 | 0.0 | 5.2 | 19.6 | 0.0 |
| II | 0.0 | 0.0 | 0.0 | 11.6 | 23.2 | 0.0 |
| II | 0.0 | 0.0 | 0.0 | 8.8 | 30.8 | 0.0 |
| II | 0.2 | 5.0 | 0.0 | 3.9 | 10.3 | 0.0 |
| II | 1.6 | 4.9 | 0.0 | 16.1 | 36.9 | 0.0 |
| II | 697.1 | 1,172.5 | 217.6 | 2,424.8 | 4,337.8 | 631.0 |
| II | 41.9 | 47.8 | 33.6 | 1,074.3 | 2,146.7 | 394.3 |
| III | 228.3 | 1,309.4 | 41.2 | 1,408.5 | 7,865.4 | 211.8 |
| III | 280.9 | 516.6 | 45.2 | 1,544.3 | 2,705.4 | 383.1 |
| III | 290.7 | 470.4 | 97.2 | 1,967.1 | 4,463.5 | 449.6 |
| III | 8,196.6 | 19,339.4 | 110.8 | 84,469.9 | 173,239.6 | 1,942.9 |
| III | 1,480.7 | 6,099.3 | 214.7 | 6,057.5 | 18,661.6 | 1,185.5 |
| III | 2,598.2 | 22,209.9 | 56.4 | 30,679.9 | 130,872.6 | 3,247.4 |
| III | 146.8 | 402.7 | 51.9 | 828.5 | 3,083.7 | 287.3 |

TABLE 20

| | | | Glc (μM) | | | Fru (μM) | | |
|---|---|---|---|---|---|---|---|---|
| Clade | Transgene | Host | Ave | Max | Min | Ave | Max | Min |
| III | AtSW14 | A. thaliana | 60.4 | 211.6 | 24.9 | 163.2 | 451.8 | 74.8 |
| III | AtSW15 | A. thaliana | 796.6 | 2,064.2 | 143.1 | 1,140.0 | 2,727.5 | 226.1 |
| IV | AtSW16 | A. thaliana | 3.1 | 14.6 | 0.0 | 0.5 | 3.0 | 0.0 |
| IV | AtSW17 | A. thaliana | 2.0 | 3.5 | 0.0 | 1.2 | 3.7 | 0.0 |
| II | OsSW05 | A. thaliana | 2.7 | 5.3 | 0.0 | 3.8 | 12.8 | 0.0 |
| III | OsSW11 | A. thaliana | 318.0 | 607.1 | 81.5 | 490.8 | 833.1 | 179.7 |
| III | OsSW12 | A. thaliana | 41.7 | 172.9 | 9.7 | 89.5 | 334.1 | 32.4 |
| III | OsSW13 | A. thaliana | 48.5 | 160.9 | 8.0 | 121.0 | 367.7 | 24.8 |
| III | OsSW13 | O. sativa | 62,407.2 | 125,776.4 | 3,917.0 | 77,858.6 | 156,842.0 | 4,650.0 |
| III | OsSW14 | A. thaliana | 37.5 | 128.5 | 10.7 | 115.6 | 460.4 | 45.5 |
| III | OsSW14 | O. sativa | 43,115.4 | 90,201.0 | 543.0 | 58,581.3 | 152,827.3 | 229.1 |
| III | OsSW15 | A. thaliana | 14.9 | 39.7 | 8.2 | 39.3 | 97.3 | 19.6 |
| III | OsSW15 | O. sativa | 33,018.8 | 246,007.1 | 197.8 | 31,135.4 | 197,244.2 | 461.9 |
| II | SlSW08 | A. thaliana | 24.8 | 27.0 | 22.6 | 17.2 | 24.2 | 10.2 |
| II | SlSW08 | O. sativa | 3,610.7 | 8,809.1 | 166.6 | 2,428.7 | 7,311.1 | 174.2 |
| II | PpSW08 | O. sativa | 6,938.7 | 15,188.5 | 1,647.2 | 4,502.9 | 12,333.7 | 1,255.4 |
| — | none | A. thaliana | 1.6 | 8.1 | 0.0 | 0.3 | 7.3 | 0.0 |
| — | none | O. sativa | 1.0 | 8.3 | 0.0 | 0.0 | 0.2 | 0.0 |

| | | | | Total Monosacharide | | |
|---|---|---|---|---|---|---|
| | Suc (μM) | | | Equivalent (μM) | | |
| Clade | Ave | Max | Min | Ave | Max | Min |
| III | 48.8 | 118.7 | 22.2 | 321.2 | 900.7 | 151.6 |
| III | 514.2 | 1,217.2 | 70.1 | 2,965.0 | 6,511.9 | 582.7 |
| IV | 0.0 | 0.0 | 0.0 | 3.5 | 14.6 | 0.0 |
| IV | 0.0 | 0.0 | 0.0 | 3.2 | 7.1 | 0.0 |
| II | 2.2 | 3.9 | 0.0 | 10.8 | 21.5 | 0.0 |
| III | 221.0 | 723.7 | 14.0 | 1,250.7 | 2,887.6 | 360.7 |
| III | 36.9 | 127.8 | 3.0 | 205.0 | 762.5 | 71.1 |
| III | 41.3 | 93.7 | 19.6 | 252.1 | 716.0 | 71.9 |
| III | 22,687.7 | 74,320.2 | 64.5 | 185,641.2 | 358,704.4 | 8,994.7 |
| III | 51.2 | 118.0 | 19.4 | 255.6 | 824.9 | 95.0 |
| III | 7,104.2 | 21,756.3 | 10.8 | 115,905.1 | 275,262.5 | 793.8 |
| III | 25.5 | 82.0 | 7.2 | 105.3 | 300.9 | 59.8 |
| III | 2,011.4 | 10,537.3 | 85.2 | 68,176.9 | 450,340.4 | 830.2 |
| II | 9.8 | 14.0 | 5.6 | 61.6 | 62.6 | 60.7 |
| II | 95.1 | 340.9 | 4.2 | 6,229.6 | 16,157.4 | 349.2 |
| II | 166.8 | 849.8 | 1.5 | 11,775.3 | 29,221.8 | 3,070.2 |
| — | 0.0 | 2.6 | 0.0 | 2.0 | 11.0 | 0.0 |
| — | 0.1 | 0.8 | 0.0 | 1.3 | 8.3 | 0.0 |

It was found that the concentration of sugar in guttation was greatly increased in Arabidopsis thaliana transformed to strongly express the DNA encoding the AtSWEET8 protein as seen in Tables 19 and 20. In particular, it was found that the concentration of sugar in guttation is greatly increased in the plants in which the DNAs encoding the AtSWEET9, AtSWEET10, AtSWEET11, AtSWEET12, AtSWEET13, AtSWEET14, and AtSWEET15 proteins classified in clade III, among the DNAs encoding the SWEET proteins, were introduced and that only in the plants in which the DNAs encoding the AtSWEET8 protein and the homologous proteins thereof, among the proteins classified in clade II, while they are not classified in clade III, the concentration of sugar in guttation can be more greatly increased, as seen in Tables 19 and 20.

Even in the wild type plants, sugar concentrations of around 50 M can be detected in discharge in some individuals. It was found that the effect of introducing the nucleic acid encoding the AtSWEET8 protein is much higher than the highest concentration detected in the wild type plants as seen in the Examples.

1-50. (canceled)

51. A transformed plant or a transformed plant cell in which a nucleic acid encoding a protein according to any of the following (a) to (c) is introduced and/or expression of a protein encoded by the nucleic acid is enhanced:
(a) a protein having the amino acid sequence set forth in SEQ ID NO: 5 or 7;
(b) a protein having an amino acid sequence having an identity of 90% or more to the amino acid sequence set forth in SEQ ID NO: 5 or 7 and having transporter activity involved in sugar transportation;
(c) a protein having an amino acid sequence encoded by a polynucleotide hybridizable with all of a polynucleotide having the nucleotide sequence set forth in any of SEQ ID NOs: 40 to 43 under stringent conditions and having transporter activity involved in sugar transportation.

52. The transformed plant or transformed plant cell according to claim 51, wherein the transformed plant is a phanerogam or derived from a phanerogam.

53. The transformed plant or transformed plant cell according to claim 52, wherein the phanerogam is an angiosperm.

54. The transformed plant or transformed plant cell according to claim 53, wherein the angiosperm is a monocot.

55. The transformed plant or transformed plant cell according to claim 54, wherein the monocot is a plant of the family Poaceae.

56. The transformed plant or transformed plant cell according to claim 55, wherein the plant of the family Poaceae is a plant of the genus Oryza.

57. The transformed plant or transformed plant cell according to claim 53, wherein the angiosperm is a dicot.

58. The transformed plant or transformed plant cell according to claim 57, wherein the dicot is a plant of the family Brassicaceae.

59. The transformed plant or transformed plant cell according to claim 58, wherein the plant of the family Brassicaceae is a plant of the genus Arabidopsis.

60. A method for producing an exudate, comprising the steps of cultivating or culturing a transformed plant or a transformed plant cell in which a nucleic acid encoding a protein according to any of the following (a) to (e) is introduced and/or expression of a protein encoded by the nucleic acid is increased; and collecting an exudate from the transformed plant or transformed plant cell:
(a) a protein having an amino acid sequence set forth in SEQ ID NO: 2, 5 or 7;
(b) a protein having an amino acid sequence having an identity of 90% or more to an amino acid sequence set forth in SEQ ID NO: 2, 5 or 7 and having transporter activity involved in sugar transportation;
(c) a protein having an amino acid sequences set forth in any of SEQ ID NOs: 3, 4, 6, 8, and 9;
(d) a protein having an amino acid sequence having an identity of 90% or more to an amino acid sequence set forth in any of SEQ ID NOs: 3, 4, 6, 8, and 9 and having transporter activity involved in sugar transportation;
(e) a protein having an amino acid sequence encoded by a polynucleotide hybridizable with all of a polynucleotide having a nucleotide sequence set forth in any of SEQ ID NOs: 1 and 40 to 43 under stringent conditions and having transporter activity involved in sugar transportation.

61. The method for producing an exudate according to claim 60, wherein the transformed plant or transformed plant cell is cultivated or cultured under conditions at a relative humidity of 80% RH or more.

62. The method for producing an exudate according to claim 60, wherein the exudate is guttation.

63. A method for producing an exudate, comprising the steps of cultivating or culturing a transformed plant or a transformed plant cell in which a nucleic acid encoding a protein having a consensus sequence comprising the following amino acid sequence: (N/S)(V/I)xxxxxFx(S/A)(1-3aa)TFxxI(V/F/M)Kx(K/R)(S/K/T)(V/T)x(D/E)(F/Y)(S/K)x(I/V/M)PY(V/I/L)x(T/A)x(L/M)(N/S)xxLW(V/T)(V/F/L)YGL(0-2aa)(V/I/F/L)xxxxxLVx(T/S)(I/V)N(A/G)xGxx(I/L)(E/H)(L/F/M/I)xY(L/I/V)x(L/I/V)(Y/F)Lxx(A/S/C)(2-4aa)(S/K/N)x(R/Q)(1-2aa)(V/I/M)xxxxxxx(L/V/I)xx(F/V/L)xx(V/I/M)xx(L/I/V)(V/T)(L/F)xx(V/I)(H/D/K)(D/S/N/G)(2-3aa)(R/K)xx(I/V/L/F)(I/V/L)Gx(L/M/I)xxx(F/L)xxxMYx(S/A)Pxx(V/A)xxxV(I/V)xx(R/K)S(V/T)(E/K)(Y/F)MPF(L/F)LS(L/F)(F/V)xF(I/L/V)N(G/A/S)xxWxxY(A/S)x(F/I/V/L)(2-3aa)Dx(F/Y)(I/V)xx(P/S)Nx(L/I)Gx(L/F/I)x(G/A)x(A/T/S)QLx(L/V)Yxx(Y/F)xx(A/S)(T/S)P and having transporter activity involved in sugar transportation is introduced and/or expression of the protein is enhanced; and collecting an exudate from the transformed plant or transformed plant cell.

64. The method for producing an exudate according to claim 63, wherein the consensus sequence comprises MVDAKQVRFIIGVIGNVISFGLFAAPAKTFWRIFKKKSVEEFSYVPYVAT(V/I)MNCMLW VFYGLPVVHKDSxLVSTINGVGLVIE(L/I)FYV(G/A)(V/L)YLxYCGHK(Q/K)NxR(K/R)(K/N)ILx(Y/F)LxxEV(V/I)xV(A/V)xI(V/I)L(V/I)TLF(V/A)(I/L)K(N/G)DFxKQTFVG(V/I)ICD(V/I)FNIAMY(A/G)(S/A)PSLAI(I/F)(T/K)VV(K/R)TKS(V/T)EYMPFLLSLVCFVNA(A/G)IWT(S/ T)YSLIFKIDxYVLASNGIGT(F/L)LALSQLIVYFMYYKSTPK(0-1aa)(E/D)KTVKPSEVEI(P/S)(A/G)T(N/E/D)RV.

65. The method for producing an exudate according to claim 63, wherein the protein having transporter activity involved in sugar transportation is an AtSWEET8 protein or a protein encoded by a homologous nucleic acid of a nucleic acid encoding the AtSWEET8 protein.

66. The method for producing an exudate according to claim 65, wherein the AtSWEET8 protein is a protein according to any of the following (a) to (c):
(a) a protein having the amino acid sequences of SEQ ID NO: 2;
(b) a protein having an amino acid sequence having an identity of 90% or more to the amino acid sequence set forth in SEQ ID NO: 2 and having transporter activity involved in sugar transportation;
(c) a protein having an amino acid sequence encoded by a polynucleotide hybridizable with all or a part of a polynucleotide having the nucleotide sequence set forth in SEQ ID NO: 1 under stringent conditions and having transporter activity involved in sugar transportation.

67. The method for producing an exudate according to claim 65, wherein the homologous nucleic acid is a nucleic acid encoding a protein according to any of the following (a) to (c):
(a) a protein having an amino acid sequence set forth in SEQ ID NO: 5 or 7;
(b) a protein having an amino acid sequence having an identity of 90% or more with an amino acid sequence set forth in SEQ ID NO: 5 or 7 and having transporter activity involved in sugar transportation;
(c) a protein having an amino acid sequence encoded by a polynucleotide hybridizable with all or a part of a polynucleotide having a nucleotide sequence set forth in any of SEQ ID NOs: 40 to 43 under stringent conditions and having transporter activity involved in sugar transportation.

68. The method for producing an exudate according to claim 65, wherein the homologous nucleic acid is a nucleic acid encoding a protein according to any of the following (a) and (b):
(a) a protein having an amino acid sequence set forth in any of SEQ ID NOs: 3, 4, 6, 8, and 9;
(b) a protein having an amino acid sequence having an identity of 90% or more to an amino acid sequence set forth in any of SEQ ID NOs: 3, 4, 6, 8, and 9 and having transporter activity involved in sugar transportation.

69. The method for producing an exudate according to claim 65, wherein the homologous nucleic acid is a nucleic acid encoding a protein according to any of the following (a) to (c):
(a) a protein having an amino acid sequence having a match of 33% or more with the amino acid sequence set forth in SEQ ID NO: 2 and having transporter activity involved in sugar transportation;
(b) a protein comprising an amino acid sequence having a match of 35% or more with the amino acid sequence of the N-terminus to a.a. 213 in the amino acid sequence set forth in SEQ ID NO: 2 as the region except the transmembrane domain and having transporter activity involved in sugar transportation;
(c) a protein comprising an amino acid sequence having a match of 37% or more with the amino acid sequence of a.a. 33 to 213 in the amino acid sequence set forth in SEQ ID NO: 2 as the region except the low homology region and the transmembrane domain and having transporter activity involved in sugar transportation.

70. The method for producing an exudate according to claim 65, wherein the homologous nucleic acid is a nucleic acid encoding a protein according to any of the following (a) to (c):
(a) a protein having an amino acid sequence having a match of 29% or more with the amino acid sequence set forth in SEQ ID NO: 5 and having transporter activity involved in sugar transportation;
(b) a protein comprising an amino acid sequence having a match of 39% or more with the amino acid sequence of the N-terminus to a.a. 205 in the amino acid sequence set forth in SEQ ID NO: 5 as the region except the transmembrane domain and having transporter activity involved in sugar transportation;
(c) a protein comprising an amino acid sequence having a match of 40% or more with the amino acid sequence of a.a. 30 to 205 in the amino acid sequence set forth in SEQ ID NO: 5 as the region except the low homology region and the transmembrane domain and having transporter activity involved in sugar transportation.

71. The method for producing an exudate according to claim 65, wherein the homologous nucleic acid is a nucleic acid encoding a protein according to any of the following (a) to (c):
(a) a protein having an amino acid sequence having a match of 30% or more with the amino acid sequence set forth in SEQ ID NO: 7 and having transporter activity involved in sugar transportation;
(b) a protein comprising an amino acid sequence having a match of 37% or more with the amino acid sequence of the N-terminus to a.a. 195 in the amino acid sequence set forth in SEQ ID NO: 7 as the region except the transmembrane domain and having transporter activity involved in sugar transportation;
(c) a protein comprising an amino acid sequence having a match of 39% or more with the amino acid sequence of a.a. 18 to 195 in the amino acid sequence set forth in SEQ ID NO: 7 as the region except the low homology region and the transmembrane domain and having transporter activity involved in sugar transportation.

72. The method for producing an exudate according to claim 60, wherein the transformed plant is a phanerogam or derived from a phanerogam.

73. The method for producing an exudate according to claim 72, wherein the phanerogam is an angiosperm.

74. The method for producing an exudate according to claim 73, wherein the angiosperm is a monocot.

75. The method for producing an exudate according to claim 74, wherein the monocot is a plant of the family Poaceae.

76. The method for producing an exudate according to claim 75, wherein the plant of the family Poaceae is a plant of the genus Oryza.

77. The method for producing an exudate according to claim 73, wherein the angiosperm is a dicot.

78. The method for producing an exudate according to claim 77, wherein the dicot is a plant of the family Brassicaceae.

79. The method for producing an exudate according to claim 78, wherein the plant of the family Brassicaceae is a plant of the genus Arabidopsis.


SEQUENCE LISTING (52 sequences)

SEQ ID NO: 1 - DNA, 720 bp, Arabidopsis thaliana
  CDS (1)..(720)
atggttgatgcaaaacaagttcgtttcatcatcggagttatcggaaatgttatctcctttggtctctttgccgcaccagcgaagactttctggaggatcttcaagaagaaatcagtggaagagttttcgtatgtgccgtacgtagcaacggtgatgaattgtatgttgtgggtgttttacggtctccctgtggttcacaaagacagtattctagtttcaaccattaatggtgttgggttagttatcgaacttttctacgttggtgtctacttgatgtactgtggtcacaagaagaaccatcgaaggaacattttgggattcttagctcttgaagttattttggtggtggctatcattcttattacgctctttgcgcttaagggtgattttgttaagcaaacatttgttggtgtgatttgcgatgtcttcaacattgctatgtatggagctccttcattggccattattaaagtggtaaaaacaaagagtgttgaatacatgccattcttgttgtctttggtctgtttcgttaatgctggaatttggactacttactcgctcatcttcaagatcgattactacgtcctcgcaagtaatgggattggaacctttttggcactttctcagttgatagtgtacttcatgtactataagtcaactccaaaggagaagacggtgaagccatcagaagttgagatttctgctacggagagggtttag

SEQ ID NO: 2 - protein, 239 aa, Arabidopsis thaliana
MVDAKQVRFIIGVIGNVISFGLFAAPAKTFWRIFKKKSVEEFSYVPYVATVMNCMLWVFYGLPVVHKDSILVSTINGVGLVIELFYVGVYLMYCGHKKNHRRNILGFLALEVILVVAIILITLFALKGDFVKQTFVGVICDVFNIAMYGAPSLAIIKVVKTKSVEYMPFLLSLVCFVNAGIWTTYSLIFKIDYYVLASNGIGTFLALSQLIVYFMYYKSTPKEKTVKPSEVEISATERV

SEQ ID NO: 3 - protein, 240 aa, Arabidopsis thaliana
MVDAKQVRFIIGVIGNVISFGLFAAPAKTFWRIFKKKSVEEFSYVPYVATVMNCMLWVFYGLPVVHKDSYLVSTINGVGLVIELFYVGVYLMYCGHKQNYRKKILLYLLGEVVSVAIIVLITLFVIKNDFIKQTFVGIICDIFNIAMYASPSLAIITVVKTKSVEYMPFLLSLVCFVNAAIWTSYSLIFKIDYYVLASNGIGTFLALSQLIVYFMYYKSTPKKEKTVKPSEVEIPATNRV

SEQ ID NO: 4 - protein, 239 aa, Capsella rubella
MVDAKQVRFIIGVIGNVISFGLFAAPAKTFWRIFKKKSVEEFSYVPYVATIMNCMLWVFYGLPVVHKDSILVSTINGVGLVIEIFYVALYLAYCGHKKNARRNILGFLILEVVAVAIIVLVTLFAIKNDFAKQTFVGVICDIFNIAMYASPSLAIFKVVRTKSTEYMPFLLSLVCFVNAAIWTSYSLIFKIDIYVLASNGIGTLLALSQLIVYFMYYKSTPKDKTVKPSEVEISGTDRV

SEQ ID NO: 5 - protein, 293 aa, Solanum lycopersicum
MANFSFILGIIGNVISILMFAAPIKTFKRIMKKKSTEDFKGIPYITTLLSTCLWTFYGLLKPGGLLVVTVNGSGAILHIIYVTLFLIYAPEPLKIQSMKLVAIIDIAFLGAVIAITLVAVHGTTRLTLVGFLCAALNIGMYAAPLAATRTVIKMKSVEYMPFFLSFFQFLNGGVWTAYAVLVKDYFIGVPNGIGFILGAAQLILYFMYYKSSPTKSTEEKGSAHLMKREIQMKDVNGAHENENSRNLHKWKSLPKPSLVRQYSEKLVKTLSNTPSSLGSHNVHDIEKGLKEAH

SEQ ID NO: 6 - protein, 243 aa, Physcomitrella patens
MFCPVCCWSGNITAICLFTSPVPTFSKIVKKKTVAEFSGIPYVCTLLNCLLWVVYGLPIVEFQVLVISINAAGCLIEFTYLALYLTYAQKSIRMKVMKVLMAVLITFIAVTILVLELVHDKKKRKLIIGTLCAVFAVGMYVSPLTVMKMVIQTRSVKYMPFLLSLFNFINGLVWFGYAFFGGIDIFIAIPNGLGALSGIAQLALYAFYRNATPRDEDEKDGPTKPTNNSIEMEKNDTYKQSNV

SEQ ID NO: 7 - protein, 253 aa, Physcomitrella patens
MLSVRVSCNFYSPTFVDIVKRKSVGDYSGIPYICTLLNCLLWVVYGLPVVELQVLVVTINAAGVVIEMIYIGLYLKNAQRSVRVKVMKVLLAVLILFTAIAVLVFVLIHDRKTRKLLVGTLCAVFGVGMYISPLAVMRLVIWTRSVEYMPFLLSLFNFINGLVWFGYAVIGHLDIFIAIPNCLGALSGVAQLSLYAYFRPATPTVRDRNEEKGNSMKWVSSSVSILVEQNDHPPLNQPCGSIEALQICEKASN

SEQ ID NO: 8 - protein, 257 aa, Physcomitrella patens
MGHVDFKVILGVLGNITAICLFASPIPTFINIVKKKSVGDYSGIPYVCTLLNCLLWVVYGLPVVEYQVLVVTINAAGCIIELIYLALYLKNAHKSIRMKVMKVLLAVLILFTLVTVIVLELIHDKKKRKLVIGTLCAVFAVGMYVSPLTVMRMVIRTRSVEYMPFLLSLFNFINGLVWFGYAFIGGLDIFIAIPNGLGALSGVAQLSLYAFYRNATPVVRDRDDVEKAKHMKPNTDSVYVQMGQNGHPPQSEANGAH

SEQ ID NO: 9 - protein, 251 aa, Physcomitrella patens
MRVAGNITASFLFLSPVPTFWRIVKSRKVDDFSGMPYLTAALNTCLWTLYGLPFVSFQVLVVTVNAAGAGLEISYIIIYLMYSEGKARMRVVKFFAVMVCGFILMTGLVLGLVDSVDTRKTILGVMGAFLGSLMYAAPLTVMRMVIQTKSVEFMPFLLSLFVFLNSTTWTIYAGVPETDLYILIPNGLGLLLGTTQLVLYAMYRGSTPRKPSLPTFSYKLAVETPPKFAPAPDSKANRPLGPGNQKAPENV

SEQ ID NO: 10 - DNA, 36 bp, Artificial Sequence
  Synthetic DNA
taatgtcgacatgaacatcgctcacactatcttcgg

SEQ ID NO: 11 - DNA, 39 bp, Artificial Sequence
  Synthetic DNA
tatgagctcttaaacttgaaggtcttgctttccattaac

SEQ ID NO: 12 - DNA, 36 bp, Artificial Sequence
  Synthetic DNA
taatgtcgacatggatgtttttgctttcaatgcttc

SEQ ID NO: 13 - DNA, 36 bp, Artificial Sequence
  Synthetic DNA
tatgagctctcacacgtaagaaacaatcaaaggctc

SEQ ID NO: 14 - DNA, 37 bp, Artificial Sequence
  Synthetic DNA
taatgtcgacatgggtgataaacttcgattatccatc

SEQ ID NO: 15 - DNA, 37 bp, Artificial Sequence
  Synthetic DNA
tatgagctcttagatcgatgaggcattgttagaattc

SEQ ID NO: 16 - DNA, 41 bp, Artificial Sequence
  Synthetic DNA
taatgtcgacatggttaacgctacagttgcgagaaacattg

SEQ ID NO: 17 - DNA, 38 bp, Artificial Sequence
  Synthetic DNA
tatgagctctcaagctgaaactcgtttagcttgtccac

SEQ ID NO: 18 - DNA, 40 bp, Artificial Sequence
  Synthetic DNA
taatgtcgacatgacggacccccacaccgcccggacgatc

SEQ ID NO: 19 - DNA, 40 bp, Artificial Sequence
  Synthetic DNA
tatgagctctcaagcctggccaagttcgattccagcattc

SEQ ID NO: 20 - DNA, 43 bp, Artificial Sequence
  Synthetic DNA
taatgtcgacatggtgcatgaacagttgaatcttattcggaag

SEQ ID NO: 21 - DNA, 41 bp, Artificial Sequence
  Synthetic DNA
tatgagctctcaaacgccgctaactcttttgtttaaatatg

SEQ ID NO: 22 - DNA, 38 bp, Artificial Sequence
  Synthetic DNA
taatgtcgacatggtgtttgcacatttgaaccttcttc

SEQ ID NO: 23 - DNA, 40 bp, Artificial Sequence
  Synthetic DNA
tatgagctcttaaacattgttaggttcttggttggtattc

SEQ ID NO: 24 - DNA, 41 bp, Artificial Sequence
  Synthetic DNA
taatgtcgacatgttcctcaaggttcatgaaattgcttttc

SEQ ID NO: 25 - DNA, 36 bp, Artificial Sequence
  Synthetic DNA
tatgagctctcacttcattggcctcaccgatccttc

SEQ ID NO: 26 - DNA, 39 bp, Artificial Sequence
  Synthetic DNA
taatgtcgacatgagtctcttcaacactgaaaacacatg

SEQ ID NO: 27 - DNA, 36 bp, Artificial Sequence
  Synthetic DNA
tatgagctctcatgtagctgctgcggaagaggactg

SEQ ID NO: 28 - DNA, 39 bp, Artificial Sequence
  Synthetic DNA
taatgtcgacatggctctcttcgacactcataacacatg

SEQ ID NO: 29 - DNA, 38 bp, Artificial Sequence
  Synthetic DNA
tatgagctctcaagtagttgcagcactgtttctaactc

SEQ ID NO: 30 - DNA, 41 bp, Artificial Sequence
  Synthetic DNA
ggaattccatatggctctaactaacaatttatgggcatttg

SEQ ID NO: 31 - DNA, 41 bp, Artificial Sequence
  Synthetic DNA
taatgtcgacttaaacttgactttgtttctggacatccttg

SEQ ID NO: 32 - DNA, 40 bp, Artificial Sequence
  Synthetic DNA
taatgtcgacatgggagtcatgatcaatcaccatttcctc

SEQ ID NO: 33 - DNA, 36 bp, Artificial Sequence
  Synthetic DNA
tatgagctctcaaacggtttcaggacgagtagcctc

SEQ ID NO: 34 - DNA, 39 bp, Artificial Sequence
  Synthetic DNA
taatgtcgacatggcagaggcaagtttctatatcggagt

SEQ ID NO: 35 - DNA, 38 bp, Artificial Sequence
  Synthetic DNA
tatgagctcttaagagaggagaggttcaacacgtgatg

SEQ ID NO: 36 - DNA, 20 bp, Artificial Sequence
  Synthetic DNA
gtaaaacgaccagtcttaag

SEQ ID NO: 37 - DNA, 17 bp, Artificial Sequence
  Synthetic DNA
caggaaacagctatgac

SEQ ID NO: 38 - DNA, 21 bp, Artificial Sequence
  Synthetic DNA
tgctgtacttgcttggtattg

SEQ ID NO: 39 - DNA, 18 bp, Artificial Sequence
  Synthetic DNA
ggaccagaccagacaacc

SEQ ID NO: 40 - DNA, 882 bp, Artificial Sequence
  Synthetic DNA
atggcaaacttctcttttattctcggtattatcggaaatgttatctctattctcatgttcgctgctcctatcaagaccttcaagaggattatgaagaaaaagtcaactgaagatttcaagggaataccttacatcactacacttttgagtacctgtctttggactttctacggtctcttaaaaccaggaggtcttttggttgtgaccgttaacggatctggtgctattttgcatataatctatgtgacactctttttaatctacgcacctgagccacttaaaatacagtctatgaagttggttgctataattgatatcgcattcctcggagctgtgatcgcaataactttagttgctgtgcacggaaccactagacttacattggttggttttctttgcgctgcattgaacattggaatgtatgctgcacctcttgctgcaacaaggactgttattaagatgaagagtgtggaatacatgccatttttcctctctttctttcaattcctcaacggaggtgtttggacagcttatgcagttcttgtgaaggattactttataggagtgcctaacggaataggtttcattttgggtgctgcacaactcatcctctacttcatgtactacaaatcttcaccaactaagtcaacagaagagaaaggaagtgctcatctcatgaagagagaaatccagatgaaagatgttaatggtgctcatgaaaatgagaactctagaaacttgcacaagtggaagagtcttcctaaaccatctttggttaggcaatactctgagaaactcgtgaagacattatcaaataccccaagttcactcggttcacataacgttcacgatattgagaaaggattaaaggaagcacactga

SEQ ID NO: 41 - DNA, 882 bp, Artificial Sequence
  Synthetic DNA
atggcaaattttagttttattctcggtatcatcggtaatgtcatcagcatcctcatgttcgcagcccctatcaagaccttcaagcggatcatgaagaagaagtccacagaggacttcaagggaatcccttacattaccacactcctgagtacctgcctctggacattttatggccttttgaagccaggaggactcctggtggtcacggtgaacggctctggggctatcctgcacatcatctacgtcactcttttcttgatctatgccccggaaccactcaagattcaaagcatgaagctggtcgccatcattgatattgcgtttcttggcgctgttatcgcaattaccttggttgccgtgcatggcacgactcggctcacactggtggggttcctctgtgccgcgctgaatatcggaatgtacgctgcaccactcgcagcaaccagaacagtgattaagatgaagtctgtcgagtatatgccattctttctttcattctttcagttcttgaacggaggtgtttggaccgcctacgcggtcctcgttaaggactatttcatcggagtcccgaacggcatcggttttattctgggtgctgcacagcttatcttgtacttcatgtactataagtccagcccaacgaagtcgactgaggaaaagggcagtgcgcaccttatgaagagggagatccagatgaaggatgtcaacggggcccacgagaacgaaaattcccgcaatctgcataagtggaagtctctccctaagccctcactggttaggcagtactccgaaaagcttgtgaagacgttgagcaacactccgtcatcactgggtagccataacgtccacgacattgaaaaggggttgaaggaagcacactga

SEQ ID NO: 42 - DNA, 762 bp, Artificial Sequence
  Synthetic DNA
atgctttcagtgagggtttcatgcaacttttattcacctacctttgtggatattgttaagaggaagagtgtgggagattattcaggaattccttatatttgtacacttttgaactgcctcttatgggttgtgtacggtcttccagttgtggaacttcaagttttggttgtgaccattaatgctgcaggtgttgtgatcgagatgatctatatcggactctacttaaagaacgctcagagatcagttagggtgaaggttatgaaagttcttttggctgtgcttatcttgtttactgctatagcagtgcttgttttcgtgttgatccatgatagaaagaccaggaaactcttagttggaactctctgtgctgtttttggagtgggaatgtatatatctcctctcgcagttatgagattagtgatttggacaaggagtgttgaatacatgccattccttttgtctctttttaatttcatcaacggattggtgtggtttggttacgctgttataggacatttggatatattcattgcaatccctaactgcctcggagctttatctggtgttgcacaactctcattatatgcttactttagacctgcaactccaacagttagagataggaatgaggagaagggtaactctatgaagtgggtttcttcaagtgtgagtattttggttgagcagaacgatcacccaccattaaatcagccttgtggttcaatagaagcactccaaatctgcgaaaaagcatcaaattga

SEQ ID NO: 43 - DNA, 762 bp, Artificial Sequence
  Synthetic DNA
atgttgtcggttcgggtttcgtgcaatttctacagccctacttttgttgatatcgtgaagcggaagagcgttggggattacagcggtattccttacatctgcaccctcctgaactgtcttttgtgggtggtctatgggctgcccgttgtggagctccaggtcctggtcgttacaattaacgccgcgggtgtggtcatcgaaatgatctacattggcctttatttgaagaatgcccaacggtcggttagagtgaaggtcatgaaggtcctcctggcggttctcattctgttcaccgctatcgcagttctcgtgtttgtcctgattcacgaccgcaagacgaggaagcttttggtcggcactctctgcgctgttttcggcgtggggatgtacatttccccgcttgccgtgatgcggctcgtcatctggaccagaagtgtggagtatatgccatttctcctgtccctcttcaactttattaatggcctggtttggttcgggtacgccgtgatcggacacctcgatatctttattgcgatcccgaactgtcttggagccttgtcaggtgtcgcgcagctttcgttgtacgcttatttcagaccagcaaccccaacagtgcgcgacaggaacgaggaaaagggaaatagcatgaagtgggtttccagctctgtgtctatcctcgtcgaacaaaatgatcatccgcccctgaaccagccctgcgggtccattgaagccctccaaatctgcgaaaaggcgtccaactga

SEQ ID NO: 44 - protein, 10 aa, Artificial Sequence
  A part of synthetic Consensus Sequence 1
XXXXXXXFXX

SEQ ID NO: 45 - protein, 34 aa, Artificial Sequence
  A part of synthetic Consensus Sequence 1
TFXXIXKXXXXXXXXXXPYXXXXXXXXLWXXYGL

SEQ ID NO: 46 - protein, 30 aa, Artificial Sequence
  A part of synthetic Consensus Sequence 1
XXXXXXLVXXXNXXGXXXXXXYXXXXLXXX

SEQ ID NO: 47 - protein, 3 aa, Artificial Sequence
  A part of synthetic Consensus Sequence 1
XXX

SEQ ID NO: 48 - protein, 25 aa, Artificial Sequence
  A part of synthetic Consensus Sequence 1
XXXXXXXXXXXXXXXXXXXXXXXXX

SEQ ID NO: 49 - protein, 57 aa, Artificial Sequence
  A part of synthetic Consensus Sequence 1
XXXXXGXXXXXXXXXMYXXPXXXXXXVXXXXSXXXMPFXLSXXXFXNXXXWXXYXXX

SEQ ID NO: 50 - protein, 30 aa, Artificial Sequence
  A part of synthetic Consensus Sequence 1
DXXXXXXNXXGXXXXXXQLXXYXXXXXXXP

SEQ ID NO: 51 - protein, 222 aa, Artificial Sequence
  A part of synthetic Consensus Sequence 2
MVDAKQVRFIIGVIGNVISFGLFAAPAKTFWRIFKKKSVEEFSYVPYVATXMNCMLWVFYGLPVVHKDSXLVSTINGVGLVIEXFYVXXYLXYCGHKXNXRXXILXXLXXEVXXVXXIXLXTLFXXKXDFXKQTFVGXICDXFNIAMYXXPSLAIXXVVXTKSXEYMPFLLSLVCFVNAXIWTXYSLIFKIDXYVLASNGIGTXLALSQLIVYFMYYKSTPK

SEQ ID NO: 52 - protein, 17 aa, Artificial Sequence
  A part of synthetic Consensus Sequence 2
XKTVKPSEVEIXXTXRV